(19) United States
(12) Patent Application Publication
(10) Pub. No.: US 2006/0019268 A1
(43) Pub. Date: Jan. 26, 2006

(54) Molecular markers of cisplatin resistance in cancer and uses thereof

(75) Inventors: Timothy C. Cheng, Toronto (CA); Stephen B. Howell, Del Mar, CA (US); Gerald Manorek, San Diego, CA (US); Charles C. Berry, San Diego, CA (US); Goli Samimi, Los Angeles, CA (US)

Correspondence Address: FULBRIGHT & JAWORSKI, L.L.P., 600 CONGRESS AVENUE, SUITE 2400, AUSTIN, TX, 78701, US

(73) Assignee: Research Development Foundation

(21) Appl. No.: 11/091,938

(22) Filed: Mar. 28, 2005

Related U.S. Application Data
(60) Provisional application No. 60/556,785, filed on Mar. 26, 2004.

(51) Int. Cl. C12Q 1/68

(52) U.S. Cl. 435/6

(57) ABSTRACT

The present invention relates generally to the fields of molecular biology and medicine. More particularly, it concerns cancer treatments and the identification and use of markers for cancer resistance to a platinum-drug based therapy therapy. In certain embodiments, the present invention provides diagnostic and/or prognostic methods involving a collection of differentially expressed genes that may be used to identify cisplatin resistance in human ovarian cancer.

This application claims the benefit of U.S. Provisional Application Ser. No. 60/556,785 filed Mar. 26, 2004, the entire disclosure of which is specifically incorporated herein by reference without disclaimer. The government owns rights in the present invention pursuant to grant number CA 78648 and CA95298 from the National Institutes of Health.

### BACKGROUND OF THE INVENTION

1. Field of the Invention

The present invention relates generally to the fields of molecular biology and medicine. More particularly, it concerns cancer treatments and markers for cancer resistance to platinum-drug based therapies (e.g., cisplatin).

2. Description of Related Art

Cisplatin is an important, and often highly effective, chemotherapeutic for the initial therapy of a variety of human cancers, but resistance often emerges quickly during treatment and the mechanisms that mediate resistance remain poorly defined. The emergence of cisplatin-resistant cancer cells in a subject both limits the clinical effectiveness of cisplatin and can severely reduce opportunities to administer an effective therapy for the cancer. Cisplatin-resistance of cancer cells thus presents a serious problem for the treatment of cancer. Certain cancerous tumors exhibit intrinsic or natural resistance to cisplatin and undergo no regression even upon initial chemotherapeutic treatment. Other cancerous tumors respond well to initial treatment but upon relapse exhibit reduced responsiveness to the drug. This type of resistance, which occurs after a course of therapy with cisplatin, is termed “acquired resistance”. The ability to quickly identify, prevent, overcome, and/or reverse cisplatin resistance would be of significant benefit for the treatment of cancer.

Although attempts have been made to identify the mechanism of cisplatin resistance, this mechanism remains to be elucidated. In various studies, cisplatin resistance has been associated with reduced intracellular accumulation of the drug, increased DNA repair function and/or increased drug detoxification by intracellular thiols (e.g. Andrews and Howell, 1990; Kelley and Rozencweig, 1989; Perez et al., 1990; and Timmer-Bosscha et al. 1992). A role for drug detoxification by intracellular thiols has been postulated due to an association of cisplatin resistance in certain cancer cell lines that display increased levels of glutathione and metallothionein (see e.g. Godwin et al. 1992 and Kelley et al. 1988).

Attempts have also been made to implicate particular genes with acquisition of a cisplatin resistant phenotype; however, this work has not resulted in the identification of a gene which can sufficiently explain or predict cisplatin resistance. For example, glutathione-S-transferase (GST) and metallothionein genes have been transfected into cell lines in an attempt to confer cisplatin resistance on the cells. GST has been reported to confer cisplatin resistance on cells but the level of increased resistance was only in the range of 1.5 to 3.0 fold (see e.g. Miyazaki et al., 1990 and Puchalski et al., 1990). Another study has reported that transfection of cells with a metallothionein gene can confer cisplatin resistance on cells but again the level of increased resistance was less than 5-fold (see Kelley et al., 1988) and other studies found no increase in cisplatin resistance upon transfection of cells with the metallothionein gene (see Morton et al., 1993 and Koropatnick and Pearson, 1993). In another study, cells transfected with the c-myc gene were reported to have acquired resistance to cisplatin but once again the level of increased resistance was very low (i.e., less than 3-fold).

Genes which may confer resistance to certain anthracycline chemotherapeutics have not been able to explain resistance to cisplatin. Multidrug resistance of tumor cells to anthracyclines (e.g. doxorubicin, epipodophyllotoxins and Vinca alkaloids) has been found to be associated with increased expression of P-glycoprotein (see Roninson et al., 1984; Riordan et al., 1985; MRP (see Cole et al., 1992). Transfection of cells with the mdr1 gene (encoding P-glycoprotein) or with the MRP gene can confer multidrug resistance on the cells (see Gros et al., 1986 and Cole, (1994). However, neither P-glycoprotein or MRP are able to confer on a cell high level resistance to cisplatin and therefore do not account for cisplatin resistance observed in tumor cells.

Thus, the art is deficient in molecular markers useful for creating diagnostic and/or prognostic tools to gauge the response of patients with cancer to cisplatin. The ability to quickly identify cancer cells that are resistant to cisplatin would significantly enhance the ability to more effectively treat the cancer and individualize a cancer therapy for a patient.

### SUMMARY OF THE INVENTION

The present invention provides improved methods to identify cancers that are resistant to a platinum-drug based therapy (e.g., cisplatin). The present invention provides genetic markers for resistance to a platinum-drug based therapy. The present invention further allows for, in certain embodiments, the individualization of a cancer therapy, monitoring the responses of a cancerous or precancerous cell to a chemotherapeutic, and screening for novel and improved cancer therapies.

One aspect of the present invention relates to a method of detecting resistance to a platinum-drug based therapy comprising assessing expression in a cell of at least one gene from group 1 or group 2; wherein group 1 comprises TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, PRO2000, GPR126, OSTF, IMAGE:924929, IMAGE:79216, and IMAGE:1473168; and wherein group 2 comprises DPH2L1, ENDO180, IFITM1, RIMS1, MHC class II suppressor, IMAGE:868555, and IMAGE:1417815; wherein increased expression of a gene from group 1 or decreased expression of a gene from group 2 indicates that the cell is resistant to the platinum-drug based therapy. Group 1 may comprise TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, IMAGE:924929, IMAGE:79216, and IMAGE:1473168. Group 2 may comprise DPH2L1, END0180, IFITM1, IMAGE:868555, and IMAGE:1417815. The cell may be a cancerous or pre-cancerous cell. In certain embodiments, the cell is a human cell. The cell may be comprised in a subject (e.g., a human subject). The method may further comprise extracting RNA from said cell. Expression of the gene may be determined using northern blot, PCR, real-time PCR, RT-PCR, Q-RT-PCR, or differential display. In certain embodiments, expression of the gene is determined using a DNA chip or a microarray. The method may further comprise determining the expression of at least a second gene whose expression may influence a cancer phenotype, wherein said second gene is not from group 1 or group 2. The method comprises determining the expression of at least two, three, four, five six, seven, eight, nine, ten or more genes from group 1 or group 2. In certain embodiments, the method comprises determining the expression of all genes from group 1 and group 2. The method may further comprise assessing the expression of metallothionein IIA in the cell.

In certain embodiments, the method comprises individualization of a cancer therapy for the subject. The individualization of a cancer therapy may comprise administering a chemotherapeutic, an anti-cancer drug (e.g., Avastin or Herceptin), a surgical therapy, or a radiation therapy to the subject. The individualization of a cancer therapy may comprise administering a platinum-drug based chemotherapeutic to the subject. The individualization of a cancer therapy may further comprise the administration of a second chemotherapeutic. The individualization of a cancer therapy may comprise avoiding the administration of a platinum-drug based chemotherapeutic to the subject. The cancer may have originated in the brain, oral cavity, upper respiratory tract, lung, breast, upper gastrointestinal tract, lower gastrointestinal tract, pancreas, liver, kidney, bladder, prostate, bone, skin, bone marrow, a lymphatic organ, testis, ovary, Fallopian tube, or peritoneum. In certain embodiments, the cancer is ovarian cancer. The cell may be comprised in a solid tumor. The cell may be metastasized. The method may comprise administration of the platinum-drug based therapy to the subject, and subsequently monitoring for resistance to the platinum-drug based therapy. The method may comprise monitoring for resistance to the platinum-drug based therapy prior to the administration of a platinum-drug based therapy to the subject. In certain embodiments, the platinum-drug based therapy is carboplatin, oxaliplatin, satraplatin, or cisplatin.

Another aspect of the present invention relates to a diagnostic kit for detecting resistance to a platinum-drug based therapy, said kit comprising probes for a group of genes comprising one or more of TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, DPH2L1, ENDO180, IFITM1, IMAGE:924929, IMAGE:79216, IMAGE:1473168, IMAGE:868555, and IMAGE:1417815. In certain embodiments, the probes are PCR primers for the group of genes. The kit may further comprise reagents for PCR reactions. The platinum-drug based therapy may be cisplatin.

Another aspect of the present invention relates to a diagnostic array for detecting cisplatin resistance, said array comprises a solid support and a plurality of diagnostic agents coupled to said solid support, wherein said diagnostic agents are used to assay the expression levels of one or more of TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, DPH2L1, ENDO180, metallothionein IIA, IFITM1, IMAGE:924929, IMAGE:79216, IMAGE:1473168, IMAGE:868555, and IMAGE:1417815. The diagnostic agents may be DNA or RNA molecules that specifically hybridize to the transcripts of said genes.

Another aspect of the present invention relates to a method for identifying a modulator of the expression of resistance to a platinum-drug based therapy comprising:(a) providing a candidate modulator; (b) contacting the candidate modulator with a cell; (c) evaluating the expression of one or more of TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, DPH2L1, ENDO180, IFITM1, IMAGE:924929, IMAGE:79216, IMAGE: 1473168, IMAGE:868555, or IMAGE: 1417815 in the cell; (d) comparing the expression measured in step (c) with the expression of the cell in the absence of said candidate modulator, wherein a difference between the expression indicates that said candidate modulator can affect resistance to a platinum-drug based therapy. The platinum-drug based therapy may be cisplatin. In certain embodiments, the cell is a cancerous cell, such as a human ovarian cancer cell.

The use of the word “a” or “an” when used in conjunction with the term “comprising” in the claims and/or the specification may mean “one,” but it is also consistent with the meaning of“one or more,” “at least one,” and “one or more than one.” As used herein “another” may mean at least a second or more.

It is contemplated that any embodiment discussed in this specification can be implemented with respect to any method or composition of the invention, and vice versa. Furthermore, compositions of the invention can be used to achieve the methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Dendrogram based on complete hierarchical clustering with average linkage of all of the 36 replicates.

FIG. 2: Histograms of the log₂(R/S) difference for each possible pairwise combination of replicates per cell line pair.

FIGS. 3A-3F: Correlation plots of log₂(R/S) for each possible pairwise combination of replicates for each cell line pair. The correlation coefficient is shown at the bottom right of each plot. The average correlation coefficient for each cell line pair is as follows: 2008 (FIG. 3A), 0.359; A2780 (FIG. 3B), 0.593; HEY (FIG. 3C), 0.647; IGROV-1 (FIG. 3D), 0.712; KF (FIG. 3E), 0.759; and UCI (FIG. 3F), 0.710.

FIG. 4: Average linkage hierarchical clustering by Euclidean distance of the average log₂(R/S) for features that passed quality control in at least 4 of 6 replicates for the 26 features that were differentially expressed in 4 of 6 cell pairs. Increased expression is seen as a more intense red and reduced expression as darker green. Missing values and lack of differential expression appear as a mid-tone grey.

FIG. 5: Image map of Pearson correlation values for the 26 features SAM identified in 4 of 6 cell pairs using log₂(R/S) for all features passing quality control in 4 of 6 replicates. The horizontal axis orders genes from left to right from highest to lowest correlation value with the other genes and the vertical axis orders genes reading downwards from highest to lowest correlation with ApoE.

FIG. 6: shows the cell cycle pathway significantly altered in 5 of 6 cell types. Darkened genes in were significantly differentially expressed (either up or down) as determined by SAM analysis.

FIG. 7: shows the pathway of benzoate degradation via CoA ligation significantly altered in 4 of 6 cell types. Darkened genes in were significantly differentially expressed (either up or down) as determined by SAM analysis.

FIG. 8: shows the pathway of butanoate metabolism significantly altered in 4 of 6 cell types. Darkened genes in were significantly differentially expressed (either up or down) as determined by SAM analysis.

FIG. 9: shows the oxidative phosphorlyation pathway significantly altered in 4 of 6 cell types. Darkened genes in were significantly differentially expressed (either up or down) as determined by SAM analysis.

FIG. 10: shows the selenoamino acid metabolism pathway significantly altered in 4 of 6 cell types. Darkened genes in were significantly differentially expressed (either up or down) as determined by SAM analysis.

FIG. 11: shows the starch and sucrose metabolism pathway significantly altered in 4 of 6 cell types. Darkened genes in were significantly differentially expressed (either up or down) as determined by SAM analysis.

### DESCRIPTION OF ILLUSTRATIVE EMBODIMENTS

The present invention provides improved methods to identify cancers that are resistant to a platinum-drug based therapy (e.g., cisplatin). The present invention provides genetic markers for resistance to a platinum-drug based therapy. The present invention further allows for, in certain embodiments, the individualization of a cancer therapy, monitoring the responses of a cancerous or precancerous cell to a chemotherapeutic, and screening for novel and improved cancer therapies.

### I. PLATINUM-DRUG BASED THERAPIES

The present invention identifies the altered expression of specific genetic markers can indicate the resistance of a cell (e.g., a cancerous or pre-cancerous cell) to a platinum-based therapy. “Platinum-based therapy”, “platinum drug-based” or “platinum-based chemotherapeutic”, as used herein, refers to any chemotherapeutic that contains platinum, such as a platinum derivative. Platinum-drug based therapies include but are not limited to: cisplatin, carboplatin, oxaliplatin, and satraplatin.

A. Cisplatin

The chemotherapeutic drug cisplatin (cis-diamminedichloroplatinum, DDP, or CDDP) was discovered to have cytotoxic properties in 1968 and is used extensively worldwide in the treatment of many tumors, in particular solid tumors such as ovarian cancer, testicular cancer and head and neck cancers. This platinum drug is thought to act by platination of DNA, thereby crosslinking DNA (both interstrand and intrastrand) and disrupting cellular processes.

Cisplatin is an effective first-line therapeutic against many types of cancer but the rapid development of resistance during therapy remains a major clinical challenge. Cisplatin is thought to kill cells predominantly by forming adducts in DNA that block transcription and replication leading to cell death. Mechanisms implicated in cellular resistance include reduced drug uptake, increased drug efflux, increased DNA repair, increased tolerance of DNA damage, and increased levels of intracellular thiols such as glutathione and metallothionein (1). More recently, the copper transporters have been found to modulate intracellular platinum levels (2). No single overarching mechanism of resistance is apparent and the consensus remains that resistance is multi-factorial in origin.

B. Other Platinum Derivatives

The markers of cisplatin resistance presented herein may further be used to predict cancer resistance to any other platinum based chemotherapy which is presently known or which may be subsequently discovered. These therapies include platinum analogs, such as carboplatin, oxaliplatin, and satraplatin. Carboplatin and oxaliplatin share similar pharmacokinetics to cisplatin (Jacobs et al., 2005). Satraplatin is another platinum derivative (Sternberg et al., 2005), and resistance to satraplatin may be predicted by methods of the present invention. Multiple platinum based chemotherapies are known in the art (Belani 2004; Farrell, 2004; Baruah et al, 2004; Hall et al., 2004; Natile et al., 2004).

### II. GENETIC MARKERS FOR RESISTANCE TO PLATINUM-DRUG BASED CHEMOTHERAPIES

Alterations in the expression of certain genes has been identified by the inventors as contributing to the resistance of a cell (e.g., an ovarian cancer cell) to a platinum-drug based therapy (e.g., cisplatinum). These genes (genetic markers) may be either up-regulated or down-regulated in cells that are resistant to a platinum-drug based therapy. The up-regulation (i.e., increased expression) of one or more of the following genes are genetic markers for resistance to a platinum-drug based therapy: TXNIP, ANXA, APOE, CLDN4, DKFZP564D0462, DKFZp761C121, F3, GADD45B, JUN, KIAA0470, MGC5254, OSTF1, PELI1, STARD4, TIMP1, TMSB10, IMAGE:924929, IMAGE:79216, and IMAGE:1473168. In certain embodiments, the up-regulation of one or more of the following genes may also contribute to the development of resistance to a platinum-drug based therapy: PRO2000, ANXA1, GPR126, and/or OSTF1. The down-regulation (i.e., decreased expression) of one or more of the following genes are genetic markers for resistance to a platinum-drug based therapy: DPH2L1, ENDO180, IFITM1, IMAGE:868555, and IMAGE:1417815. In certain embodiments, the down-regulation of the expression of one or more of the following genes may also contribute, in certain embodiments, to the development of resistance to a platinum-drug based therapy: RIMS1, and/or MHC class II suppressor. Down-regulation of the MHC class II suppressor gene has been shown to contribute to resistance to a platinum-drug based therapy in some, but not all, of the studies presented herein; nonetheless, the inventors presently believe that it may contribute to resistance to a platinum-drug based therapy. Evaluation of the expression of these genes in a cancer may be used to assess the likelihood of response to subsequent treatment with a platinum-containing agent at any point in a patient's course (e.g., before the start of any therapy, at various points during a course of therapy, or at the end of a course of therapy, either primary or secondary). “Resistance to a platinum-drug based therapy”, “Resistance to a platinum-based therapy” or “Resistance to a platinum-based chemotherapeutic”, as used herein, is defined as displaying at least one change in the expression of a gene as described above. Changes in the expression of two, three, four, five, six, seven, eight, nine, ten, or more different genes as described above can also indicate resistance to a platinum-drug based therapy.

### III. INDIVIDUALIZATION OF CANCER THERAPIES

The present invention provides a more thorough understanding of the mechanisms that mediate cisplatin resistance as well as molecular markers of resistance to platinum-drug based therapies which may be used for individualizing therapy. Given the fact that cisplatin causes substantial toxicity, avoiding administration of this drug to patients whose tumors have little chance of responding is important and may be preferred if cancer cells of the patient display expression patterns consistent with resistance to platinum-drug based therapies.

A cancer therapy may be individualized in many ways. For example, a cancer therapy may be individualized upon evaluation of cisplatin resistance by altering the dose and/or type of chemotherapeutic being administered, or by the administration of an additional therapy, such as radiotherapies (e.g., radiation therapy or radionuclide therapy) or surgery.

“Therapy individualization” or “individualization of a cancer therapy”, as used herein, is defined as the modification or alteration of a cancer therapy or strategy for the treatment of cancer subsequent to evaluation of expression of the genetic markers of the present invention which may predict resistance to a platinum-drug based therapy.

### IV. CANCER

Certain embodiments of the present invention may be directed towards diagnosing cancer, predicting responses to certain treatments (e.g., cisplatin resistance) for cancer, and monitoring responses to treatments of cancer. Normal tissue homeostasis is a highly regulated process of cell proliferation and cell death. An imbalance of either cell proliferation or cell death can develop into a cancerous state (Solyanik et al., 1995; Stokke et al., 1997; Mumby and Walter, 1991; Natoli et al., 1998; Magi-Galluzzi et al., 1998). For example, cervical, kidney, lung, pancreatic, colorectal and brain cancer are just a few examples of the many cancers that can result (Erlandsson, 1998; Kolmel, 1998; Mangray and King, 1998; Mougin et al., 1998). In fact, the occurrence of cancer is so high that over 500,000 deaths per year are attributed to cancer in the United States alone.

Changes in gene expression are associated with many, if not most, forms of cancer. The maintenance of cell proliferation and cell death is at least partially regulated by proto-oncogenes. A proto-oncogene can encode proteins that induce cellular proliferation (e.g., sis, erbB, src, ras and myc), proteins that inhibit cellular proliferation (e.g., Rb, p16, p19, p21, p53, NF1 and WT1) or proteins that regulate programmed cell death (e.g., bcl-2) (Ochi et al., 1998; Johnson and Hamdy, 1998; Liebermann et al., 1998). However, genetic rearrangements or mutations to these proto-oncogenes, results in the conversion of a proto-oncogene into a potent cancer causing oncogene. Often, a single point mutation is enough to transform a proto-oncogene into an oncogene. For example, a point mutation in the p53 tumor suppressor protein results in the complete loss of wild-type p53 function (Vogelstein and Kinzler, 1992) and acquisition of “dominant” tumor promoting function. In certain embodiments of the present invention, a cell may be evaluated for expression levels of genes that may not indicate cisplatin resistance but which may indicate another attribute of a cancerous cell (e.g., the type of cancer, or resistance to another chemotherapeutic); these genes may be simultaneously with, subsequently to, or previous to evaluation of the genes of the present invention which indicate resistance to a platinum derivative (e.g., cisplatin)

Cancer cells that may be identified as developing or as having developed resistance to cisplain by the methods of the present invention include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human ovarian cancer. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### V. NUCLEIC ACID DETECTION AND GENE EXPRESSION ANALYSIS

Differential expression of genes which may confer resistance to a platinum therapy (e.g., cisplatin) may be evaluated by a variety of techniques. Multiple techniques are well known in the art regarding the analysis of gene expression. Gene expression may be evaluated by assessing levels of a species of RNA in a cell (e.g., using microarray analysis or real-time PCR) or by assessing the amount of a protein in a cell (e.g., via a Western blot or via mass spectroscopy). In certain embodiments, the identification of a mutation in the DNA of a gene (e.g., identification of a null mutation which prevents the expression of a gene) may also be used to evaluate gene expression. All techniques that are presently known, or which may be subsequently discovered, for the evaluation of gene expression are contemplated for use with the present invention. Techniques for evaluating gene expression include microarray analysis, differential display, PCR, RT-PCR, Q-RT-PCR, Northern blots, Western blots, and Southern blots.

A. Hybridization

Hybridization is a technique well known in the art that is often used in experiments concerning nucleic acids. The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences involved with the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl2, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl2, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions. selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

Amplification of nucleic acids is another technique that may be used in certain embodiments of the present invention. Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to specific genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR amplification procedure (RT-PCR) may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may-then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR™” (Frohman, 1990; Ohara et al., 1989).

Of particular interest to the present invention is the use of reverse transcription (RT), reverse transcription PCR (RT-PCR), and qualitative reverse transcription PCR (Q-RT-PCR). As is well known in the art, RNA can be reverse transcribed to DNA (e.g., cDNA) via a reverse transcriptase. Many products are commercially available for performing reverse transcription. RT-PCR is well known in the art and is often used to amplify cDNA sequences. In some instances, these sequences are specific to a single gene; however, for the purposes of microarray analysis, typically multiple primers are used to insure that essentially all cDNA species are amplified. The fluorescence-based Q-RT-PCR, also known as “real-time reverse transcription PCR”, is widely used for the quantification of mRNA levels and is a critical tool for basic research, molecular medicine and biotechnology. Q-RT-PCR assays are easy to perform, capable of high throughput, and can combine high sensitivity with reliable specificity (Bustin, 2002).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In other embodiments, detection is by Northern blotting and hybridization with a labeled probe. Northern blotting provides a way to measure mRNA. The techniques involved in Northern blotting are well known to those of skill in the art (Trayhurn, 1996). A cDNA labelled with ³²P is the most commonly used probe, although other methods (including non-radioactive detection methods) also exist.

In other embodiments of the invention, gene expression may be analyzed using mass spectroscopy. Since its inception and commercial availability, the versatility of matrix assisted laser desorbtion ionization time-of-flight mass spectrometry (MALDI-TOF-MS) has been demonstrated convincingly by its extensive use for qualitative analysis. MALDI-TOF-MS has been employed for both applications relating to proteins (e.g., the characterization of synthetic polymers, peptides, recombinant proteins, and protein analysis) as well as for DNA and oligonucleotide sequencing (Miketova et al., 1997; Faulstich et al, 1997; Bentzley et al., 1996).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In particular, the inventors anticipate that MALDI-TOF-MS may be used to observe expression of RNA isolated from a cancerous or pre-cancerous cell in order to identify genes that differentially expressed and which may cause or affect (e.g., cisplatin resistance) a cancerous phenotype. Also, in another embodiment of the present invention, RNA from a cancerous or pre-cancerous cell could be reverse transcribed to cDNA, and this cDNA could be subsequently analyzed by matrix-assisted laser desorption/ionization (MALDI) techniques such as MALDI-TOF-MS.

MALDI-TOF-MS has been used for many applications, and many factors are important for achieving optimal experimental results (Xu et al., 2003). Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result. With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

Several different MALDI approaches may be used in certain embodiments of the present invention. For example, certain MALDI techniques may be used to determine specific nucleotide polymorphisms and/or for genotyping (Blondal et al., 2003; Marvin et al., 2003; Pusch et al., 2003; Tost et al., 2002; Sauer et al., 2002). In particular, these techniques may be employed in an embodiment of the present invention by genotyping and/or detecting polymorphisms in RNA and/or DNA obtained from a cancerous cell.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Other Assays for Nucleic Acid Detection

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substititution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

E. Differential Display

One embodiment of the present invention involves the use of differential display. Differential display allows a method for detecting mRNA and evaluating gene expression. Techniques involving differential display are well known in the art (Stein and Liang, 2002; Liang, 2002; Broude, 2002).

F. DNA Chips and MicroArrays

In certain embodiments of the present invention, gene expression indicative of cisplatin resistance is evaluated using a DNA chip and/or a microarray. DNA arrays and gene chip technology provides a means of rapidly screening a large number of DNA samples for their ability to hybridize to a variety of single stranded DNA probes immobilized on a solid substrate. Specifically contemplated are chip-based DNA technologies such as those described by Hacia et al. (1996) and Shoemaker et al. (1996). These techniques involve quantitative methods for analyzing large numbers of genes rapidly and accurately. The technology capitalizes on the complementary binding properties of single stranded DNA to screen DNA samples by hybridization. Pease et al. (1994); Fodor et al. (1991). Basically, a DNA array or gene chip consists of a solid substrate upon which an array of single stranded DNA molecules have been attached. For screening, the chip or array is contacted with a single stranded DNA sample which is allowed to hybridize under stringent conditions. The chip or array is then scanned to determine which probes have hybridized. In a particular embodiment of the instant invention, a gene chip or DNA array would comprise probes specific for chromosomal changes evidencing the development of a neoplastic or preneoplastic phenotype. In the context of this embodiment, such probes could include synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. No. 5,861,242 and 5,578,832 which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal, for example, luminescence. The target nucleic acid may be immobilized onto the integrated microchip that also supports a phototransducer and related detection circuitry. Alternatively, a gene probe may be immobilized onto a membrane or filter which is then attached to the microchip or to the detector surface itself. In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules.

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellous membranes, the DNA probes are spotted onto the membranes. A UV light source (Stratalinker,™ Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

### VI. SCREENING ASSAYS

The present invention has many applications for use in screening assays. For example, the present invention could be used to evaluate changes in expression produced by a drug. In this application, RNA from a cancerous or precancerous cell could be obtained and analyzed from a control subject and a subject that has been exposed to a drug. Differences in gene expression could be used to determine if the drug has commercial value. For example, if a drug results in the up-regulation of expression of genes associated with apoptosis, then the drug may have value for treating cancer. Additionally, if the drug results in the down-regulation of a molecular marker associated with platinum-drug based therapy resistance, then this drug may be useful for a combination therapy with the platinum-drug based therapy to treat cancer. In certain embodiments, the drug may be continuously administered to a cancer cell or to a patient with cancer; in other embodiments, the drug may be administered repeatedly at intervals or only a single time.

The present invention further comprises methods for identifying modulators of the expression of resistance to a platinum-drug based therapy. These assays may comprise screening of large libraries of candidate substances; alternatively, the assays may be used to focus on particular classes of compounds selected with an eye towards structural attributes that are believed to make them more likely to function more effectively as chemotherapeutics.

To identify a modulator of resistance to a platinum-drug based therapy, one generally will determine the expression of genetic markers of resistance to a platinum-drug based therapy in the presence and absence of the candidate substance, a modulator defined as any substance that alters function. For example, a method generally comprises:

(a) providing a candidate modulator;

(b) admixing the candidate modulator with an isolated compound or cell, or a suitable experimental animal;

(c) measuring one or more characteristics of the compound, cell or animal in step (c); and

(d) comparing the characteristic measured in step (c) with the characteristic of the compound, cell or animal in the absence of said candidate modulator,

wherein a difference between the measured characteristics indicates that said candidate modulator is, indeed, a modulator of the compound, cell or animal.

Assays may be conducted in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit or enhance expression of a target. The candidate substance may be a protein or fragment thereof, a small molecule, or even a nucleic acid molecule. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on gene expression in cancerous cells (e.g., altering the expression of gene markers of resistance to a platinum therapy). Regardless of the type of inhibitor or activator identified by the present screening methods, the effect of the inhibitor or activator by such a compound results in alteration of gene expression in cancerous cells (e.g., altering the expression of gene markers of resistance to a platinum therapy) as compared to that observed in the absence of the added candidate substance.

B. In Cyto Assays

The present invention also contemplates the screening of compounds for their ability to modulate markers of resistance to a platinum based therapy in cells. Various cell lines can be utilized for such screening assays, including cells specifically engineered for this purpose. Many lines of cancerous cells have been produced, and it is envisioned that any of these cell lines may be used. Additionally, cancerous cells may be obtained from a subject and used in subsequent testing.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

C. In Vivo Assays

In vivo assays involve the use of various animal models, including transgenic animals that have been engineered to have specific defects (e.g., a particular sensitivity to certain kinds of cancer), or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that alters expression of genetic markers of resistance to a platinum therapy. In these embodiments, the present invention is directed to a method for determining the ability of a candidate substance to induce gene expression of genetic markers of resistance to a platinum therapy in cancerous cells, generally including the steps of: administering a candidate substance to the animal; and determining the ability of the candidate substance to alter the expression of one or more characteristics of regulation of a genetic marker for resistance to a platinum-drug based therapy.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site (e.g., direct injection into a tumor).

Determining the effectiveness of a compound in vivo may involve a variety of different criteria. Also, measuring toxicity and dose response can be performed in animals in a more meaningful fashion than in in vitro or in cyto assays.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cells and culture. Six human ovarian carcinoma cell lines (2008, A2780, HEY, IGROV-1, KF28 and UCI 107) were used in this study. Sublines stably resistant to DDP (2008/C13*5.25, A2780/CP70, HEY C2, IGROV-1/CP, KFr13 and UCI CPR) had been prepared from each parental line by repeated in vitro exposure to DDP as previously described (Mishima et al., 2002). All cell lines were maintained in drug-free RPMI-1640 medium (GIBCO, Inc.) with 10% fetal calf serum (GIBCO, Inc.) at 37° C. in a humidified atmosphere containing 5% CO2. The degree of resistance of each subline was determined at the time RNA was harvested using a clonogenic assay with continuous drug exposure as previously described (Mishima et al., 2002).

Expression profiling. cDNA microarrays were purchased from the Stanford Functional Genomics Facility (www.microarray.org) and contained 43,200 elements representing ˜29,593 genes as estimated by association with UniGene clusters. When the cell cultures reached ˜80% confluence they were lysed with a guanidine isothiocyanate buffer (4 M guanidine isothiocyanate (Gibco, Inc.), 25 mM sodium acetate pH 5.5 (Ambion), 0.5% sarkosyl (Fisher Scientific) and 0.1 M 2-mercaptoethanol (Gibco, Inc.). Total RNA was pelleted through a cesium chloride (Gibco, Inc.) step gradient and reverse transcribed into cDNA with a 2:1 ratio of aminoallyl-dUTP (Sigma) to dTTP (Sigma). cDNA from 3 of the 6 pairs of replicates were labeled as Cy3 (sensitive) and Cy5 (resistant) (Amersham); for the remaining 3 replicates the labeling was reversed. Five hundred ng of Cy3 and Cy5-labeled cDNA were hybridized to the cDNA microarrays for 18 hours at 42° C. The arrays were washed 4 times with neat sodium citrate buffer (SSC) containing 0.1% SDS, twice with 1×SSC, once with 0.1×SSC, and spun dry.

Microarray scanning and quality assurance. Features on the microarrays were located and Cy3 and Cy5 fluorescence intensities were analyzed with GenePix Pro 3.0 software (Axon Instruments, Inc.) and a GenePix 4000A scanner (Axon Instruments, Inc.). The data sets were imported into Microsoft Excel spreadsheets for analysis of the quality of each feature. Four parameters were used to assess the quality of each feature, and features were excluded for any of the following conditions: diameter<50 um; ≧50% pixels saturated in both channels; <54% of the pixels with an intensity greater than the median background intensity plus one standard deviation in either channel; and, if flagged by GenePix as ‘not found’ or ‘absent’ or manually flagged as ‘bad’. The log2(Cy3/Cy5) ratios for replicates 1-3 and log2(Cy5/Cy3) for replicates 4-6, hereafter referred to as the log2(R/S) values, were calculated for each feature and then normalized in the R statistical environment using a within-print tip group normalization method based on locally weighted lowess regression as proposed by Yang et al. (2002). For details of the approach to evaluation of dye-bias, see ‘Significance Analysis of Microarray’ algorithm below.

Analysis of reproducibility. Four approaches were taken to assess reproducibility: 1) complete linkage hierarchical clustering of all 36 arrays; 2) correlation plots of log2(R/S) for each possible pairwise combination of replicates within each cell line pair; 3) histograms of the difference in log2(R/S) between each possible pairwise combination of replicates within each cell line pair; and, 4) determination of how many features with a significantly higher or lower expression level would be missed per cell line pair if only 4 or 5 replicates had been performed instead of 6. Complete linkage hierarchical clustering using Euclidean distance and a heatmap representation for all 36 arrays after quality assurance and normalization was carried out in the R statistical environment (Ihaka and Gentleman, 1996 as were the correlation and histogram plots for the pairwise comparisons of the log2(R/S) values. To determine how many differentially expressed features would be missed if less than 6 replicates had been performed, SAM was applied to all features that had passed quality assurance in all 6 replicates, followed by features that had met quality assurance criteria in at least 4 and 5 replicates. The average number of SAM-identified genes for each possible combination of 4 or 5 replicates was then subtracted from the total number of genes found from the 6 replicate analysis.

Identification of genes of interest using Significance Analysis of Microarrays (SAM). The SAM algorithm works as a Microsoft Excel add-in (www-stat.stanford.edu/˜tibs/SAM/index.html)and calculates a “d” score or modified t-statistic for the normalized log₂(R/S) of each feature. This is the mean log₂(R/S) divided by the standard error to which a constant value was added. The addition of a constant value gives the tests more power on average and de-emphasizes large “d” score values that arise from genes whose expression level is near zero (Storey and Tibshirani, 2003). The cutoff for significance is determined by the tuning parameter δ, which is chosen by the user based on the estimated False Discovery Rate (FDR). In these studies the value of δ was always chosen so that the estimated FDR would be very low so that of all clones ‘discovered’, the number of those falsely discovered is expected to be 1.0 or less. After filtering the data sets to include only those features for which a log₂(R/S) value was available in at least 4 of the 6 replicates, each pair of isogenic cell line pairs was subjected to SAM (Tusher et al., 2001) with permutations honoring the pairing of resistant/sensitive members of each pair within each replication. SAM analyses were carried out on the data sets from each cell line pair separately and additional SAM analyses were carried out across all 6 pairs. First the average log₂(R/S) was determined for each feature for the 6 replicates for a given cell pair. Those features for which an average log₂(R/S) was not available for all 6 pairs were discarded. In addition, the mean of the normalized log₂(R/S) ratio for each feature across all 6 replicates was calculated for each cell pair and SAM was run across all 6 cell pairs using these 6 mean values. For evaluation of dye bias between the 2 groups of differentially labeled samples (replicates 1-3 and 4-6), an unpaired two-class SAM analysis was done using all log₂(R/S) data that had passed quality control setting the false discovery rate to 1.

Statistical techniques. The data consist of 6 replications comparing sensitive and resistant members of 6 pairs of isogenic cell lines. The SAM analyses generated lists of genes deemed to be significantly up- or down-regulated in the resistant member of each isogenic pair, and these lists were used to tabulate the combinations of cell lines in which each gene was differentially expressed and the number of genes in which exactly 2, 3, 4 or 5 pairs showed differential expression. These tables were then used to calculate the expected number of genes deemed differentially expressed in 2, 3, 4, or 5 cell pairs under the hypothesis of no association of differentially expressed genes between cell lines. The probability that a gene deemed differentially expressed in k cell pairs would be differentially expressed in another pair was calculated.

Ontology and pathways analysis. All SAM-identified genes for features that had passed quality control in 4 out of 6 replicates in each cell line were used for the ‘in and out’ of category calculations wherein a hypergeometric p-value was determined given the null hypothesis that the ensemble of SAM-identified genes were not associated with a functional or biochemical relationship as defined by categories in the Kyoto Encyclopedia of Genes and Genomes (www.kegg.org) and the Gene Ontology Consortium (www.geneontology.org). Categories identified were then further winnowed down using a criterion for low false discovery. All scripts were written in the R statistical environment (Ihaka and Gentleman, 1996)

Real-time confirmation of differential expression. Quantitative real time PCR assays were established for 6 of the 27 genes differentially expressed in at least 4 of the 6 cell pairs. These 6 were selected on the basis of their linkage to biochemical mechanisms that could conceivably modulate DDP sensitivity. Differential expression of MT2A, STARD4, F3, ANXA1, CLDN4, and TIMP1 were evaluated in the cell lines using real-time PCR.

### Example 2

### Markers for Cisplatin Resistance in Human Ovarian Cancer

This Example presents genes consistently differentially expressed in pairs of isogenic cisplatin (DDP)-sensitive and resistant human ovarian carcinoma cell lines using cDNA microarrays. Most attempts to use microarray-based expression profiling to identify genes associated with a drug-resistant phenotype have relied on comparisons between independent cell lines or tumor samples. Although seemingly always successful in identifying genes differentially expressed in these different samples, the confidence that these genes are really markers of resistance has been compromised by the fact that most studies employed data derived from just one or two independently isolated RNA samples per tumor, or relied on an arbitrarily chosen threshold for identifying genes as being of interest. The present invention provides genes associated with the DDP-resistant phenotype based on a different approach designed to improve confidence by: 1) comparing multiple isogenic pairs of sensitive and stably resistant cells; 2) using a large number of independent replicates; and, 3) requiring that genes pass a rigorous test of statistical significance in order to be considered associated with the resistant phenotype.

Cisplatin sensitivity. Colony formation assays were used to determine the sensitivity of the 6 pairs of DDP-sensitive and resistant cell lines to the cytotoxic effect of DDP. The results, presented in Table 1, indicate that the difference in DDP sensitivity, as measured by the ratio of IC₅₀ values, ranged from 2.2-fold for the KF28-KFr13 pair to 11.9-fold for the A2780-A2780/CP70 pair.

TABLE 1
Cisplatin IC₅₀ values as determined by colony formation assay

| Parental cells | IC₅₀, μM* | Resistant cells | IC₅₀, μM* | Fold-resistant |
|---|---|---|---|---|
| 2008 | 0.70 | 2008/C13*5.25 | 1.10 | 1.6 |
| A2780 | 0.21 | A2780/CP70 | 2.50 | 11.9 |
| HEY | 0.76 | HEY C2 | 4.60 | 6.1 |
| IGROV-1 | 0.30 | IGROV-1/CP | 1.08 | 3.6 |
| KF28 | 0.18 | KFr13 | 0.40 | 2.2 |
| UCI 107 | 1.00 | UCI CPR | 8.00 | 8 |

*Continuous drug exposure

Quality assurance and evaluation of dye-effect. RNA was isolated from each member of the 6 pairs of cell lines, converted to cDNA, labeled with Cy3 or Cy5 and hybridized to the microarrays containing 43,200 elements representing ˜29,593 genes as estimated by their association with UniGene clusters. Four parameters were used to assess the quality of each feature: diameter, saturated pixels, intensity of features, and the number of features flagged by the scanning software. The average number of features that passed quality assurance ranged from 24.7%-38.0%; considering all replicates for all 6 pairs of cell lines a total of 13,228 features (32.3%) met the criteria.

A total of 256 features (0.6%) demonstrated a dye bias as detected by “Significance analysis of microarrays” (SAM) (Tusher et al., 2001). Among these, an average of only 36.4% were also identified by SAM analysis as exhibiting a significant degree of differential expression. Conversely, among the features that were identified by SAM analysis as exhibiting differential expression, none exhibited dye-bias as determined by two-class unpaired SAM occurred in 4 out of 6 cell pairs.

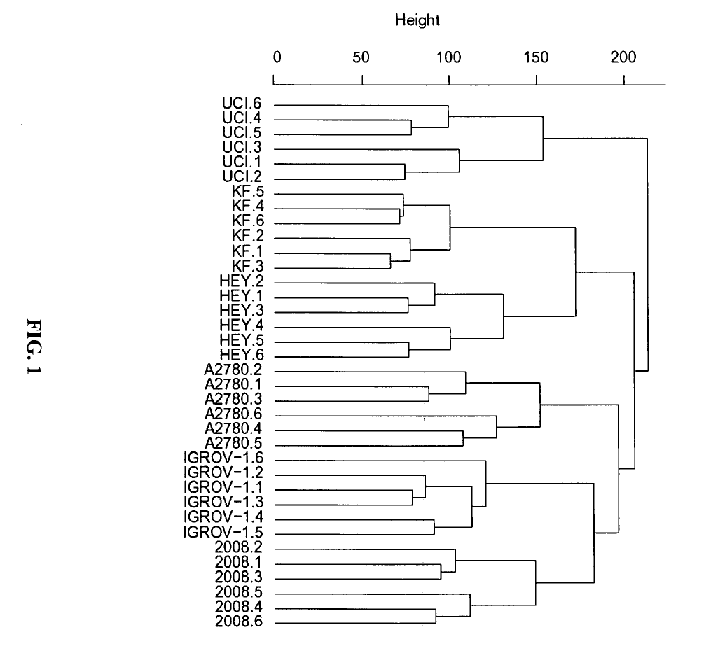

Analysis of reproducibility. One of the major challenges with the use of microarrays is to distinguish valid signals from background noise. In order to assess the reproducibility of the results and to generate data with a high signal-to-noise ratio, 6 independent replicates were performed for each cell line pair such that there were a total of 36 hybridizations when all 6 pairs were analyzed. The first assessment of reproducibility was based on hierarchical cluster analysis of all 36 samples. As shown in FIG. 1, the 6 replicates in each cell line pair merged together in the tree structure before the different cell line pairs merged with the single exception of replicate 6 from the IGROV-1 pair and replicate 4 from the UCI pair. Thus, the expression profile for a replicate of any given cell pair was more closely related to replicates from the same cell pair than to replicates from another cell pair.

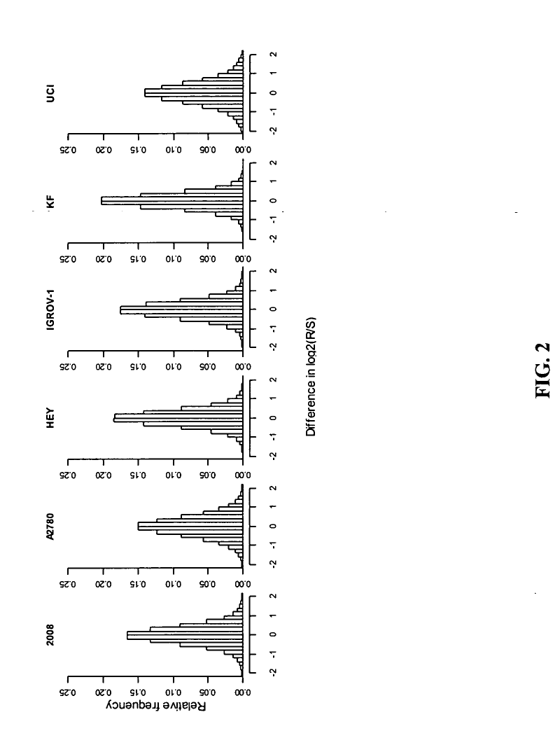

The second approach taken to the analysis of reproducibility was to examine histograms of differences in the log₂(resistant/sensitive) fluorescence ratio (log₂(R/S)). A histogram was produced for each cell line pair using the following technique. For each gene, all possible pairwise differences among the 6 replicate log₂(R/S) were calculated. A histogram was generated from all non-missing log₂(R/S) differences for all genes. The frequency in each bin was divided by the total number of log₂(R/S) differences to give the relative frequency. The resulting histograms are plotted for each pair of DDP-sensitive and resistant cell lines in FIG. 2. In these plots, better reproducibility is reflected by tall narrow histograms. Based on inspection of these histograms, the trend in reproducibility is KF>HEY>2008>IGROV-1>A2780>UCI.

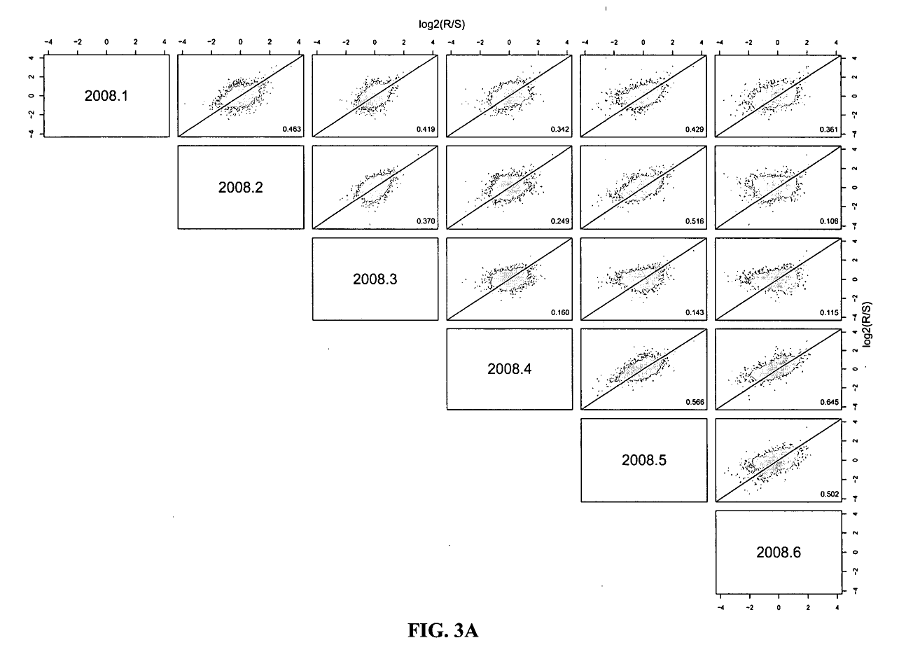

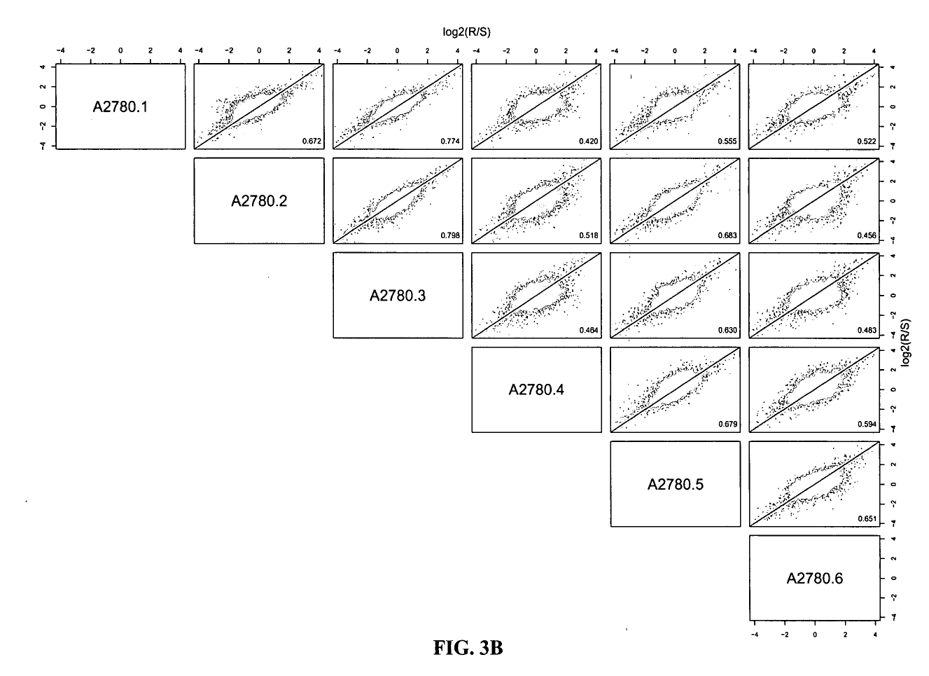

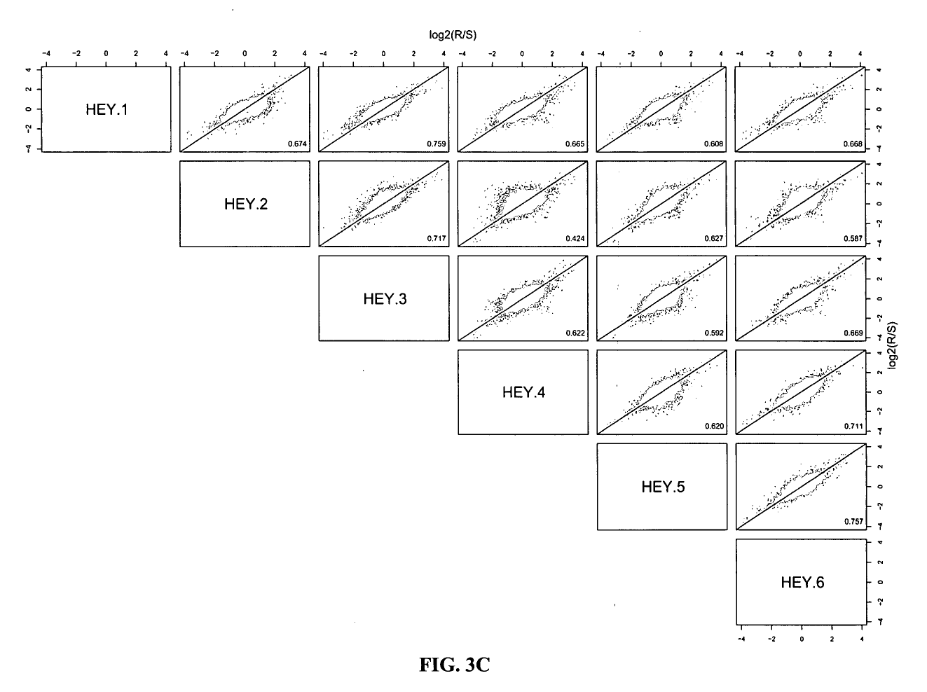

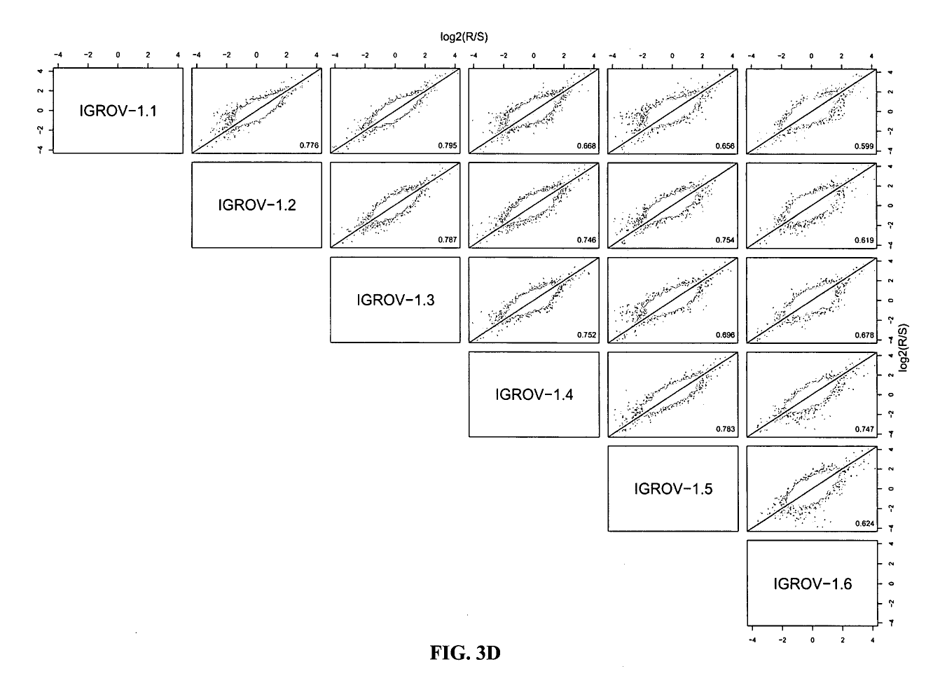

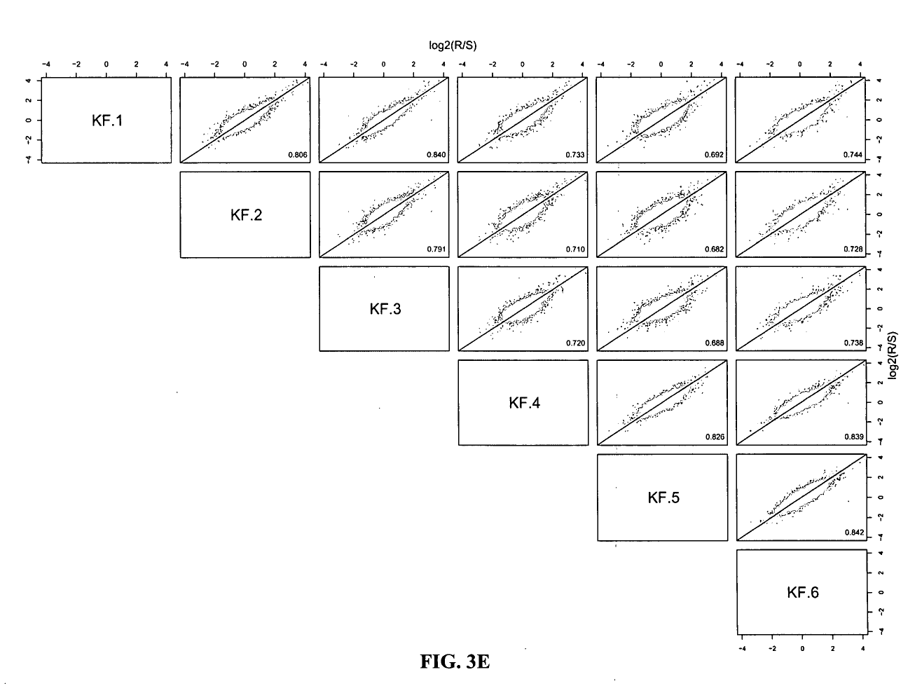

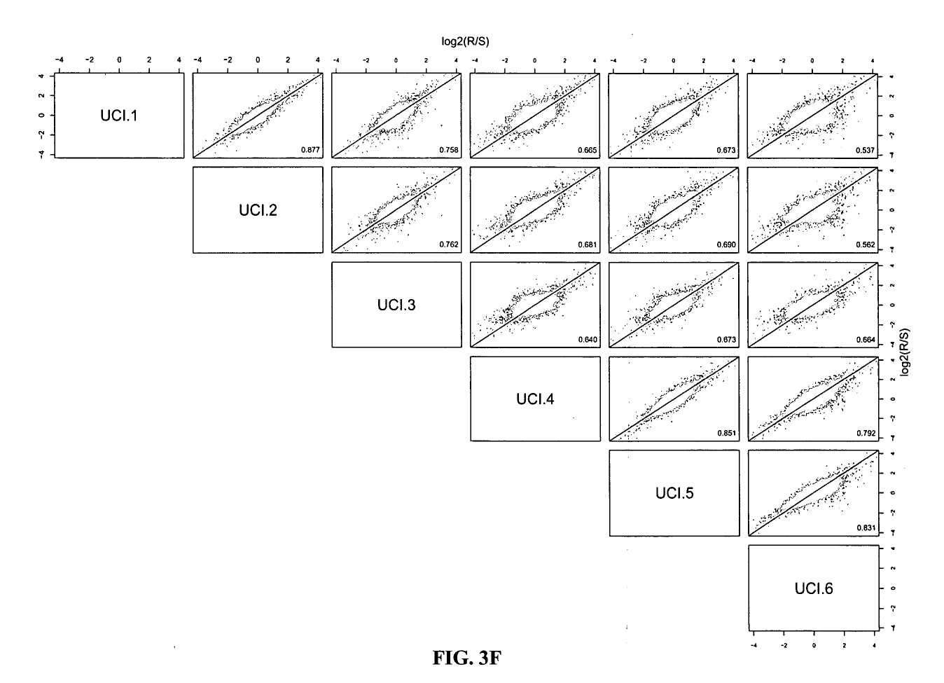

The third approach used to assess reproducibility was to construct scatterplots of the log₂(R/S) values for each feature for each possible pairwise combination of replicates within a cell line pair. If the results of any 2 replicates were exactly the same, all the data points would lie on a line with a slope of 1.0, while increasing differences between 2 replicates would be indicated by increasing degrees of scatter around this line. These plots are presented in FIGS. 3A-F; the correlation coefficient is shown at the bottom right of each plot. The averages of the correlation coefficients for all the cell pairs was 0.71. This analysis yielded that same rank order for reproducibility as the histograms of the log₂(R/S) ratio differences.

The question of how many replicates are needed to identify most of the genes that are really differentially expressed between the sensitive and resistant cell lines is important to the design of future studies. Therefore, the inventors evaluated how many of the genes that were found to be statistically significantly differentially expressed when all 6 replicates were considered would have been missed if only 4 or 5 replicates had been performed on each cell pair. SAM analysis was carried out for each cell line pair on the features that had passed quality assurance in all 6 replicates. The numbers of features discovered in the analyses are presented in Table 2; they ranged from a low of 488 features for the 2008 and 2008/C13*5.25 pair to a high of 3,797 features for the KF28 and KFr13 pair. A SAM analysis was then performed on all the features that had passed quality assurance in all 4 of all possible combinations of 4 replicates, and on all 5 of all possible combinations of 5 replicates.

TABLE 2
The number of features expressed at significantly higher
or lower levels in the cisplatin-resistant cell pairs.

| | Features | Features | | Features | | Total number of | |
|---|---|---|---|---|---|---|---|
| | analyzed | with a higher | | with a lower | | differentially | |
| Cell line pair | by SAM* | expression level | | expression level | | expressed features | |
| 2008, 2008/C13*5.25 | 11655 | 282 | (2.5%) | 206 | (1.8%) | 488 | (4.3) |
| A2780, A2780/CP70 | 14996 | 674 | (4.5%) | 718 | (4.8%) | 1392 | (9.3%) |
| HEY, HEY/C2 | 13692 | 476 | (3.5%) | 609 | (4.4%) | 1085 | (7.9) |
| IGROV-1, IGROV-1/CP | 14299 | 1411 | (9.9%) | 1449 | (10.1%) | 2860 | (20.0%) |
| KF28, KFr13 | 13461 | 2080 | (15.5%) | 1717 | (12.8%) | 3797 | (28.3%) |
| UCI107, UCICPR | 9666 | 829 | (8.6%) | 758 | (7.8%) | 1587 | (16.4%) |
| Mean | 12962 | 959 | (7.4%) | 909 | (6.9%) | 1868 | (14.3%) |

*Number of features that passed quality assurance criteria in at least 4 of the 6 replicates

As shown in Table 3, the striking finding was that an average of 24.7% of the genes discovered by SAM when all 6 replicates were considered would have been missed if only 5 replicates had been performed, and an average of fully 38.8% would have been missed if only 4 replicates had been performed. This finding mandates the use of a large number of replicates in future studies aimed at comprehensively identifying truly differentially expressed genes using cDNA arrays.

TABLE 3
The number and percentage of features with higher and lower expression levels
that would have been missed had only 4 or 5 replicates been performed

| | | No. missed features | | | Percent missed features | | |
|---|---|---|---|---|---|---|---|
| | | | Higher | Lower | | Higher | Lower |
| | No. of | | expression | expression | Total | expression | expression |
| Cell Type | replicates | Total | level | level | (%) | level (%) | level (%) |
| 2008, | 5 | 132 | 46 | 86 | 29.6 | 20.1 | 39.6 |
| 2008/C13*5.25 | 4 | 223 | 79 | 144 | 49.8 | 34.4 | 65.9 |
| A2780, | 5 | 349 | 185 | 165 | 31.5 | 37.0 | 27.0 |
| A2780/CP70 | 4 | 485 | 270 | 215 | 43.8 | 54.1 | 35.3 |
| HEY, | 5 | 262 | 119 | 143 | 20.3 | 23.9 | 23.4 |
| HEY C2 | 4 | 358 | 162 | 196 | 27.8 | 32.5 | 32.2 |
| IGROV-1, | 5 | 837 | 411 | 425 | 34.7 | 34.2 | 35.2 |
| IGROV-1/CP | 4 | 1268 | 634 | 634 | 52.7 | 52.8 | 52.6 |
| KF28, | 5 | 926 | 520 | 406 | 27.3 | 26.3 | 28.5 |
| KFr13 | 4 | 759 | 199 | 561 | 31.5 | 16.5 | 46.4 |
| UCI 107, | 5 | 298 | 134 | 164 | 25.6 | 26.6 | 24.9 |
| UCI CPR | 4 | 334 | 143 | 198 | 28.8 | 28.3 | 30.2 |
| Mean | | | | | 33.6 | 32.2 | 36.8 |

Identification of genes of interest using SAM. SAM was used to identify genes that were significantly differentially expressed within each cell line pair. In this analysis each feature on the array was required to meet the quality assurance criteria in at least 4 of the 6 replicates. The average number of features analyzed by SAM across the 6 cell pairs was 12,962; an average of 959 were found to have higher expression and 909 lower expression levels in the DDP-resistant sublines. Thus, for each cell pair a relatively large number of genes met the statistical criteria of being differentially expressed. However, the cross-tabulation presented in Table 4 and Table 5 indicated that very few of these were significantly differentially expressed in common across multiple cell pairs.

TABLE 4
The number of features in common between cell lines
that had significantly higher levels of expression

| 2008, | A2780, | HEY, | IGROV-1, | KF28, | UCI 107, | Number of |
|---|---|---|---|---|---|---|
| 2008/C13*5.25 | A2780/CP70 | HEY C2 | IGROV-1/CP | KFr13 | UCI CPR | features |
| • | • | • | | • | • | 1 |
| • | • | • | | • | • | 1 |
| | | • | • | • | • | 1 |
| | • | | • | • | • | 8 |
| • | | • | | • | • | 2 |
| | • | • | • | | • | 2 |
| • | • | | • | | • | 2 |
| • | • | • | | | • | 1 |
| • | | • | • | • | | 1 |
| • | • | • | | • | | 2 |
| | | | • | • | • | 18 |
| | | • | | • | • | 3 |
| | • | | | • | • | 6 |
| • | | | | • | • | 2 |
| | | • | • | | • | 2 |
| | • | | • | | • | 26 |
| • | | | • | | • | 3 |
| • | • | | | | • | 2 |
| | | • | • | • | | 4 |
| | • | | • | • | | 15 |
| • | | | • | • | | 8 |
| | • | • | | • | | 8 |
| • | | • | | • | | 8 |
| • | • | | | • | | 12 |
| | • | • | • | | | 3 |
| • | | • | • | | | 3 |
| • | • | | • | | | 3 |
| | | | | • | • | 68 |
| | | | • | | • | 75 |
| | | • | | | • | 11 |
| | • | | | | • | 69 |
| • | | | | | • | 14 |
| | | | • | • | | 147 |
| | | • | | • | | 73 |
| | • | | | • | | 66 |
| • | | | | • | | 65 |
| | | • | • | | | 20 |
| | • | | • | | | 86 |
| • | | | • | | | 21 |
| | • | • | | | | 5 |
| • | | • | | | | 9 |
| • | • | | | | | 9 |
| | | | | | • | 449 |
| | | | | • | | 1561 |
| | | | • | | | 963 |
| | | • | | | | 316 |
| | • | | | | | 347 |
| • | | | | | | 113 |

TABLE 5
The number of features in common between cell lines
that had significantly lower levels of expression

| 2008, | A2780, | HEY, | IGROV-1, | KF28, | UCI 107, | Number of |
|---|---|---|---|---|---|---|
| 2008/C13*5.25 | A2780/CP70 | HEY C2 | IGROV-1/CP | KFr13 | UCI CPR | features |
| | • | | • | • | • | 3 |
| | • | • | | • | • | 1 |
| | • | • | • | | • | 1 |
| | | | • | • | • | 6 |
| | | • | | • | • | 2 |
| | • | | | • | • | 8 |
| • | | | | | • | 1 |
| | | • | • | | • | 2 |
| | • | | • | | • | 6 |
| • | | | • | | • | 12 |
| | • | • | | | • | 3 |
| • | | • | | | • | 3 |
| • | • | | | | • | 4 |
| | | • | • | • | | 2 |
| | • | | • | • | | 11 |
| | • | • | | • | | 7 |
| • | | • | | • | | 2 |
| • | • | | | • | | 2 |
| | • | • | • | | | 5 |
| • | • | | • | | | 1 |
| | | | | • | • | 45 |
| | | | • | | • | 78 |
| | | • | | | • | 12 |
| | • | | | | • | 75 |
| • | | | | | • | 14 |
| | | | • | • | | 75 |
| | | • | | • | | 107 |
| | | • | | • | | 38 |
| • | | | | • | | 6 |
| | | • | • | | | 21 |
| | • | | • | | | 47 |
| • | | | • | | | 34 |
| | • | • | | | | 36 |
| • | | • | | | | 12 |
| • | • | | | | | 5 |
| | | | | | • | 440 |
| | | | | • | | 1401 |
| | | | • | | | 1145 |
| | | • | | | | 393 |
| | • | | | | | 464 |
| • | | | | | | 110 |

There were no features that were differentially expressed at either a higher or lower level in all 6 pairs or even in 5 of the 6 cell pairs. There were 22 features (20 genes) whose expression was significantly increased in 4 of 6 the pairs, and only 5 features (5 genes) whose expression was significantly decreased in 4 of the 6 resistant sublines. The gene names corresponding to these up and down-regulated features are presented in Table 6 and Table 7. Additional information about the IMAGE number in the clone ID column can be found at the website of the I.M.A.G.E Consortium (image.llnl.gov/image/). When SAM was run across all 6 cell pairs using the mean log₂(R/S) derived from the 6 replicates of each cell pair, metallothionein 2A was the only gene that passed the statistical threshold.

TABLE 6
The feature number, clone identifiers, gene symbols and gene names of the
22 features corresponding to 20 genes up-regulated in 4 of 6 cell pairs.

| N | Clone ID | Gene Symbol | Gene Name |
|---|---|---|---|
| 12574 | IMAGE: 1554367 | TXNIP | Thioredoxin interacting protein |
| 14387 | IMAGE: 488488 | TXNIP | Thioredoxin interacting protein |
| 35178 | IMAGE: 208718 | ANXA1 | Annexin A1 |
| 33376 | IMAGE: 753610 | APOE | Apolipoprotein E |
| 5382 | IMAGE: 346510 | CLDN4 | Claudin 4 |
| 38086 | IMAGE: 259884 | DKFZP564D0462 | Hypothetical protein DKFZp564D0462 |
| 21311 | IMAGE: 290162 | DKFZp761C121 | Hypothetical protein DKFZp761C121 |
| 24341 | IMAGE: 1928791 | F3 | Coagulation factor III |
| | | | (thromboplastin, tissue factor) |
| 42503 | IMAGE: 825461 | GADD45B | Growth arrest and DNA-damage-inducible, beta |
| 35087 | IMAGE: 726035 | JUN | v-Jun sarcoma virus 17 oncogene homolog (avian) |
| 34836 | IMAGE: 609377 | KIAA0470 | KIAA0470 gene product |
| 40101 | IMAGE: 280375 | MGC5254 | Hypothetical protein MGC5254 |
| 28879 | IMAGE: 700302 | OSTF1 | Osteoclast stimulating factor 1 |
| 24762 | IMAGE: 416676 | PELI1 | Pellino homolog 1 (Drosophila) |
| 2042 | IMAGE: 235909 | STARD4 | START domain containing 4, sterol regulated |
| 35082 | IMAGE: 713696 | TIMP1 | Tissue inhibitor of metalloproteinase 1 (erythroid |
| | | | potentiating activity, collagenase inhibitor) |
| 41478 | IMAGE: 840788 | TMSB10 | Thymosin, beta 10 |
| 37772 | IMAGE: 1473171 | TXNIP | Thioredoxin interacting protein |
| 4395 | IMAGE: 924929 | No gene symbol | ESTs, weakly similar to putative protein |
| | | | [Arabidopsis thaliana] [A. thaliana] |
| 12939 | IMAGE: 79216 | No gene symbol | Homo sapiens cDNA FLJ33834 fis, clone |
| | | | CTONG2004264, moderately similar to |
| | | | NEUROBLAST DIFFERENTIATION |
| | | | ASSOCIATED PROTEIN AHNAK |
| 30572 | IMAGE: 1473168 | No gene symbol | EST |

TABLE 7
The feature number, clone identifiers, gene symbols
and gene names of the 5 features corresponding to
5 genes down-regulated in 4 of 6 cell pairs.

| | | Gene | |
|---|---|---|---|
| N | Clone ID | Symbol | Gene Name |
| 30691 | IMAGE: 878468 | DPH2L1 | Diptheria toxin resistance |
| | | | protein required for |
| | | | diphthamide biosynthesis- |
| | | | like 1 (S. cerevisiae) |
| 38803 | IMAGE: 235882 | ENDO180 | Endocytic receptor |
| | | | (macrophage mannose |
| | | | receptor family) |
| 10149 | IMAGE: 509641 | IFITM1 | Interferon induced |
| | | | transmembrane protein |
| | | | 1 (9-27) |
| 5232 | IMAGE: 868555 | No gene | EST |
| | | symbol | |
| 22436 | IMAGE: 1417815 | No gene | Homo sapiens cDNA |
| | | symbol | FLJ38407 fis, clone |
| | | | FEBRA2008859 |

Ontology/pathway analysis. One reason for identifying genes that are differentially expressed in resistant cells is to use these as signposts to point out biochemical mechanisms or cellular functions that mediate DDP resistance. The 20 genes differentially expressed in at least 4 of the 6 cell pairs were examined to determine whether they were associated more frequently than would be expected by chance alone with one of the biochemical pathways defined by the Kyoto Encyclopedia of Genes and Genomes (www.KEGG.org) or with one of the ontological categories defined by the Gene Ontology Consortium (www.geneontology.org). Under the null hypothesis that the genes identified by SAM were not meaningfully associated with known pathways or functional categories, those that had a hypergeometric p-value <0.05 were identified. After a further stringent filtering of these candidate categories for low false discovery, none were found to contain a disproportionate number of the genes identified as differentially expressed by SAM analysis.

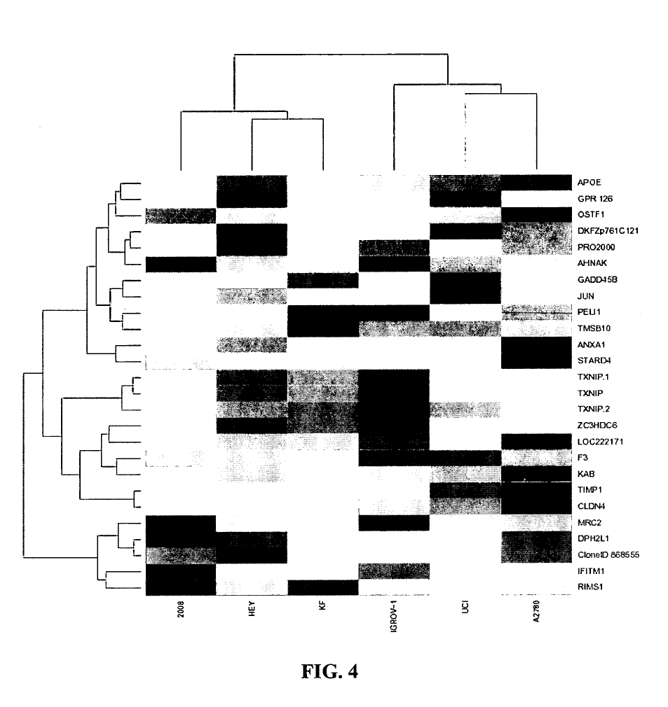

Classification of cell pairs based on expression of genes identified by SAM analysis. The 6 pairs of cell lines were clustered on the basis of the Euclidean distance of the average log₂(R/S) for the 27 features that were identified by SAM analysis as differentially expressed in 4 of 6 cell pairs. As shown in FIG. 4, the 6 cell pairs resolved into two groups consisting of the KF, HEY, and 2008 pairs and the A2780, IGROV-1, and UCI pairs. The order of the merges in the tree did not correspond to the degree of DDP resistance in each cell pair. Thus, although FIG. 4 suggests the existence of 2 separate classes of resistant cells, these classes were not distinguished by their degree of DDP-resistance.

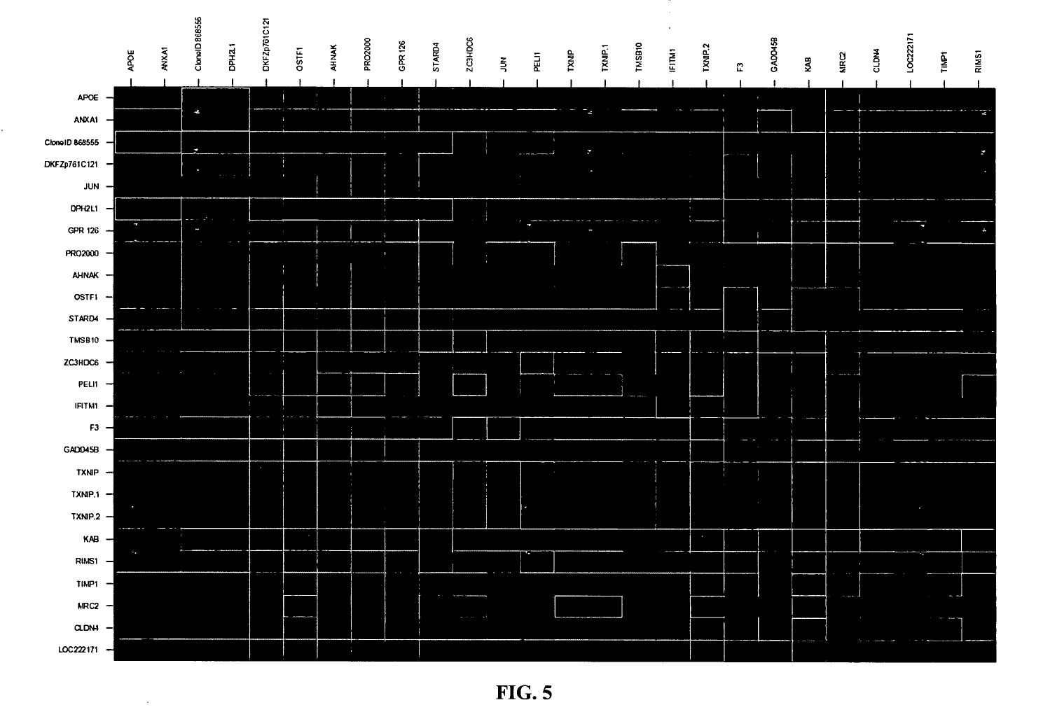

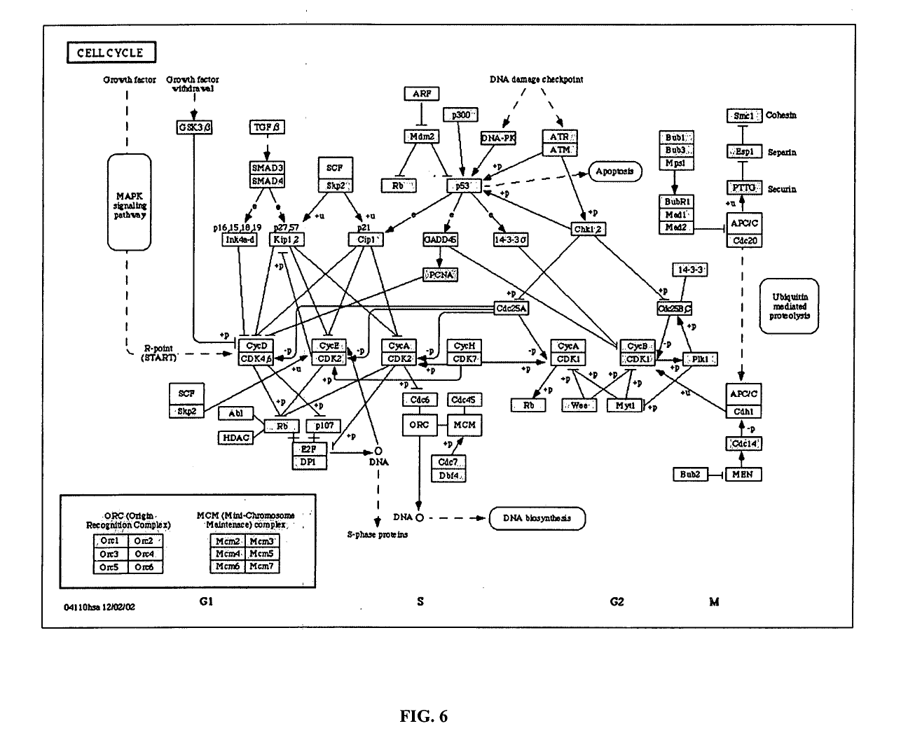

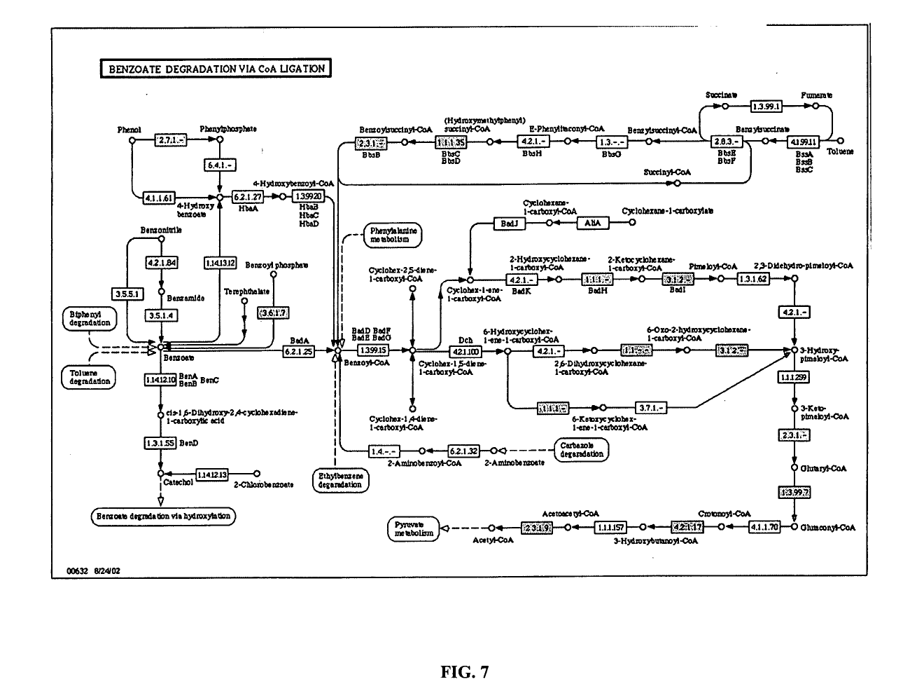

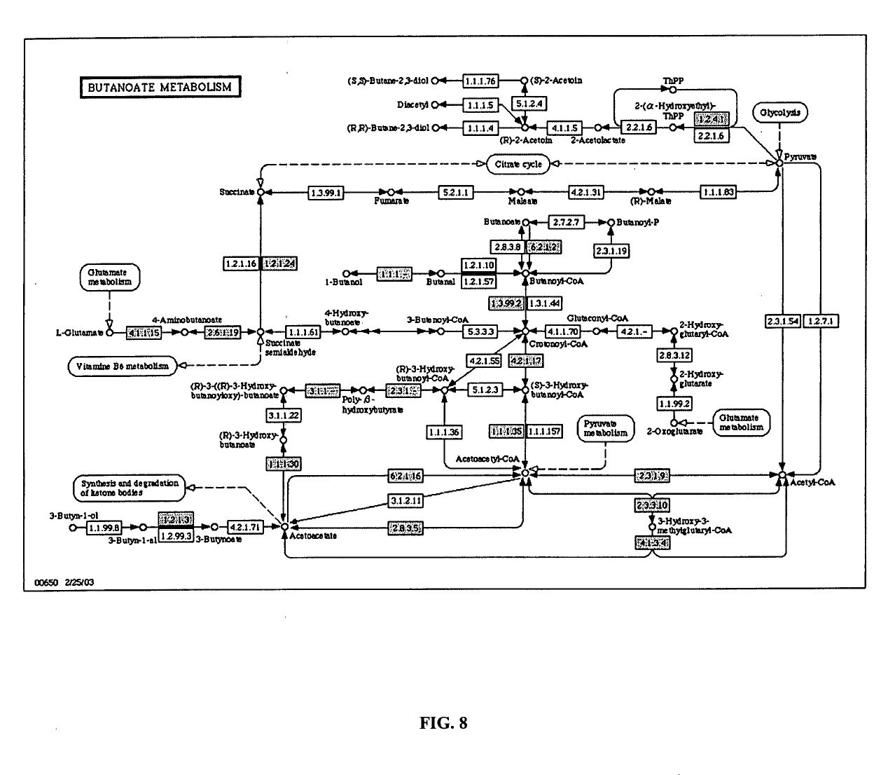

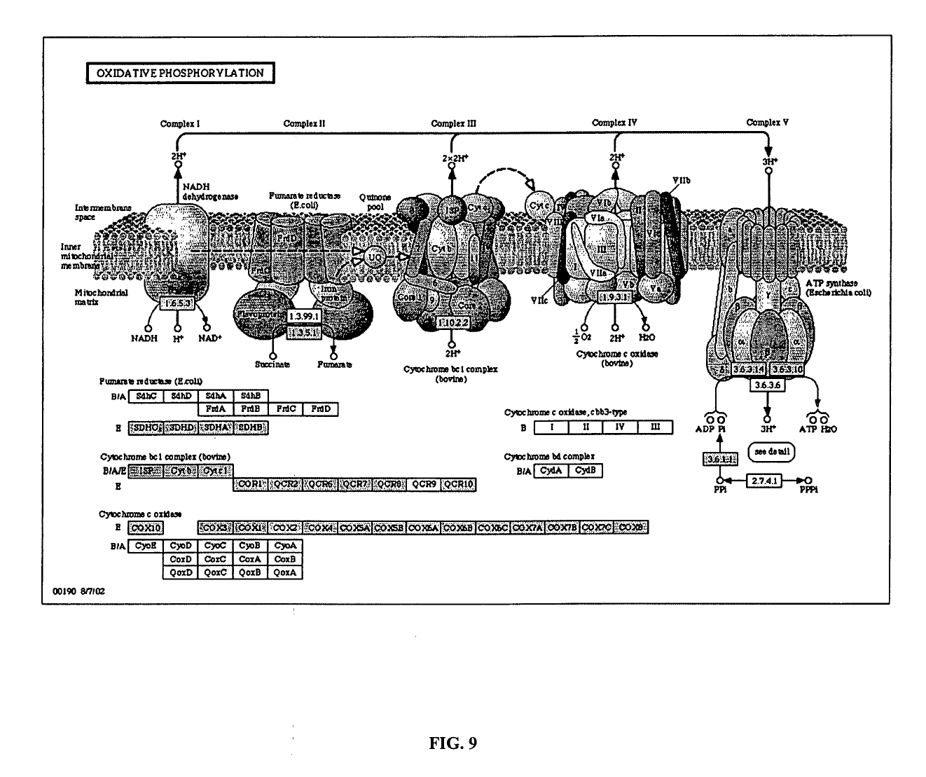

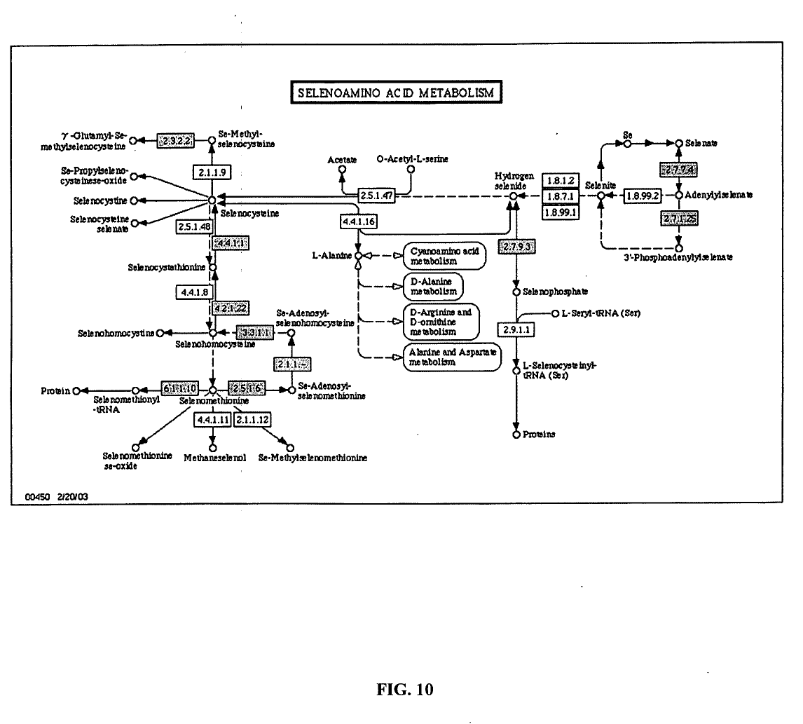

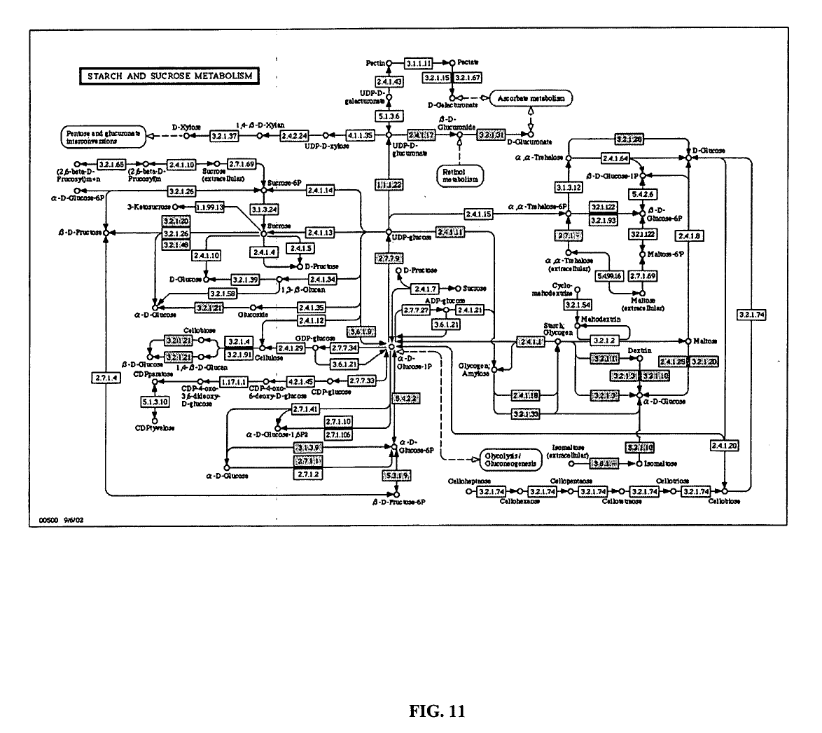

Correlation between the expression of genes associated with DDP resistance. Having identified a set of 25 genes associated with the DDP-resistant phenotype, it was of interest to determine the extent to which the degree of differential expression of any one gene was correlated with the degree of differential expression of every other gene. FIG. 5 shows a heat map of the Pearson coefficient for the correlation of each gene with every other gene in the set. Within this set there are several genes whose degree of differential expression was highly correlated with that of multiple other genes. For example, the degree of differential expression of ApoE was correlated with a large number of other genes and had the highest mean correlation coefficient. In contrast, the degree of differential expression of CLDN4 was correlated with the degree of differential expression only 2 other genes; it was positively correlated with TIMP1 and negatively correlated with KAB. This analysis indicates that, for a given increment of differential expression of one gene, there are other genes that exhibit a similar degree of differential expression across the 6 cell pairs, suggesting that they function in a parallel manner with respect to the resistant phenotype.

TABLE 8
Enumeration of features in given subsets of FIG. 5*

| | | % >1.5 fold | % all | % all features |
|---|---|---|---|---|
| | % SAM | that are | features | that are |
| Cell Line | yes >1.5-fold | SAM yes | that are >1.5 | SAM yes |
| 2008 | 2.05 | 1.17 | 7.31 | 4.19 |
| A2780 | 6.04 | 3.66 | 15.31 | 9.28 |
| HEY | 3.59 | 2.00 | 14.23 | 7.92 |
| IGROV-1 | 6.50 | 6.85 | 18.98 | 20.00 |
| KF | 6.45 | 8.96 | 20.32 | 28.21 |
| UCI | 2.61 | 0.87 | 16.75 | 5.61 |
| Mean | 4.54 | 3.92 | 15.48 | 12.53 |

*Using all features that passed quality control in at least 4 of 6 replicates

Real-time PCR confirmation of differential expression. Quantitative real time PCR assays were established for 6 of the 27 genes differentially expressed in at least 4 of the 6 cell pairs. These 6 were selected on the basis of their linkage to biochemical mechanisms that could conceivably modulate DDP sensitivity. Differential expression of MT2A, STARD4, F3, ANXA1, CLDN4, and TIMP1 was confirmed in the cell lines using real-time PCR.

Discussion. Differential expression of a given gene can arise from a genetic or epigenetic change specific to that gene, secondary effects of mutations in other genes that mediate resistance, or non-specific effects of random mutations in genes unrelated to the resistant phenotype. To sort these out, one must identify the differentially expressed genes and have a high level of confidence in this identification and this was the primary goal of this study. Regardless of the functional relevance of any particular gene, the validity of the signal is paramount, particularly if a given gene or ensemble of genes is to be used for molecular classification. This highlights the importance of analyzing the consistency with which a gene is differentially expressed both within multiple replicates from a given cell pair and across multiple pairs of sensitive and resistant cell lines. Furthermore, a large number of believably differentially expressed genes is needed to landmark known biochemical pathways and cellular functional categories that can lead to the identification of mechanisms. In these studies, the inventors approached these challenges by using 6 cell pairs and a large number of replicates in which RNA was independently isolated from separate flasks of cells growing under identical conditions, processed independently, and then analyzed in separate hybridization reactions.

The quality of each array and its features is a crucial factor in searching for genes of interest using cDNA microarrays. The use of multiple replicates permitted a rigorous assessment that disclosed that reproducibility varied significantly in different cell pairs despite the fact that all were grown under identical conditions and analyzed with identical procedures. Reproducibility across the 6 replicates was very good for the KF, IGROV-1, and HEY cell line pairs and acceptable for the A2780 and UCI cell line pair. The reproducibility for the 2008 and UCI cell line pairs was poor; however, a careful review of the technical aspects of the experiments performed with the 2008 and UCI cells did not reveal any candidate discrepancies and the source of the additional variance in these pairs remains unidentified. Interestingly, these pairs also demonstrated the smallest number of differentially expressed features (488 for 2008 and 537 for UCI), suggesting that multiple replicates of high reproducibility are necessary for efficient identification of differentially expressed genes. These analyses of reproducibility now provide a benchmark against which future microarray experiments using the Stanford microarrays can be compared.

Even when comparisons were made between stably resistant cell lines growing under highly standardized conditions, the noise associated with this cDNA microarray data was high. A large number of replicates were required in order to identify most of the significantly differentially expressed genes. The fact that fully 38.3% of the genes that were identified by SAM as being significantly differentially expressed would have been missed if only 4 instead of 6 replicates had been used has implications for future studies. It is important to find as many of these genes as possible, since false negative results in some cell line pairs will make genes that are found in other pairs seem to be the result of mutations unrelated to resistance. Further, the ability to identify cellular functions or biochemical pathways that are altered in the resistant cells increases when multiple genes are identified that share a pathway or function. Although expensive, there appears to be little alternative to the use of a large number of biologically independent replicates in future studies directed at identifying consistently differentially expressed genes.

One of the major challenges in the analysis of cDNA microarray data is to develop a strategy for judging the statistical significance of a given level of differential expression given the multiple comparisons intrinsic to the microarray approach. In these studies the Significance Analysis of Microarrays (SAM) strategy developed by Tusher et al. (2001) was used. The parameters of this analysis were set such that the median number of features identified in error (false positive rate) was ˜1. This represents a very stringent threshold, but it is noteworthy that this approach nevertheless identified a large number of differentially expressed genes within each cell pair. Thus, using these cDNA arrays, it was not difficult to identify genes that met the SAM criteria of significant differential expression even in the case where the variance across the 6 replicates was high as in 2008 and UCI cell pairs.

An improvement in the identification of real signals through the use of more replicates and SAM analysis can be seen through a comparison of features that are identified by SAM versus those that are identified by simply requiring that the expression ratio exceed some threshold, for example a ratio of 1.5. Only 3.9% of the features that exhibited an expression ratio of >1.5 were identified by SAM as being significantly differentially expressed indicating that reliance on an expression ratio >1.5 would yield 96.1% false positives. Conversely, only 4.5% of the features identified as significantly differentially expressed by SAM had expression ratios that exceeded 1.5. Thus, if one relied on an expression ratio of >1.5 only that majority of the features of potential interest would have been missed.

It is the genes that are differentially expressed in a large number of cell pairs that are of greatest interest with respect to understanding mechanisms that mediate DDP resistance. However, despite the large number of genes differentially expressed in each cell pair, none of these were consistently differentially expressed in all 6, or even in 5 of the 6, cell pairs. A total of 27 features (corresponding to 25 genes) were differentially expressed in 4 of 6 cell pairs; 20 of these genes were up-regulated and 5 were down-regulated. This suggests one of two conclusions: either the mechanisms of resistance in each pair are largely unique to that pair; or, the vast majority of the genes exhibiting differential expression are not primary determinants of the resistant phenotype. Clustering of the 6 cell pairs based only on the genes identified as significantly differentially expressed by SAM analysis provided some evidence for two DDP-resistant phenotypes within which there are additional cell pair-specific alterations.

The primary approach taken in these studies was to identify the genes that were statistically significantly differentially expressed within each cell pair using SAM analysis, and then ask how many of these genes were consistently differentially expressed in multiple cell pairs. Among the 25 genes differentially expressed in 4 of 6 isogenic pairs there are a number whose protein products are known to be involved in functions that might reasonably be expected affect the ability of DDP to kill the cell including DNA/cellular-damage response (GADD45B, c-JUN), cell-surface interactions and signaling (claudin 4, apolipoprotein E, annexin A1), lipid transport (STARD4), and cell growth and differentiation (thymosin β10, tissue factor) as well as cell-cycle arrest and growth inhibition (thioredoxin interacting protein). An alternative approach is to average the log₂(R/S) for each feature across the 6 replicates for each cell pair, and then use this mean value to perform a SAM analysis across the 6 pairs. This alternative approach identified only a single gene, metallothionein 2A, as being differentially expressed. With the exception of metallothionein 2A, it is noteworthy that neither approach identified any of the other genes previously implicated in DDP resistance, including those involved in nucleotide excision repair, DNA-damage recognition, DNA mismatch repair, apoptosis, or DDP transport. Elevated levels of metallothioneins have been found in some DDP-resistant cell lines and these sulfur-rich proteins are thought to be involved in sequestering DDP by chelation through thiol groups so that interaction of the drug with key cellular targets (Holford et al., 2000; Yang et al., 1998).

Although the resistant phenotype might result from large changes in the expression of a small number of important genes, it might also be the result of subtle changes in an ensemble of coordinately regulated genes that function in a common biochemical or signaling pathway. This possibility was investigated by considering all the genes known to be associated with pathways or cellular functions as defined in the Kyoto Encyclopedia of Genes and Genomes and the Gene Ontology Consortium databases and applying a Fisher exact in/out of category calculation and a Wilcoxon rank sum test using a Komolgorov-Smirnov statistic to SAM-identified genes that passed quality control in at least 4 of 6 replicates. The Komolgorov-Smirnov statistic was considered necessary since the usual reference distributions for these test statistics do not apply as groups of genes tend to be jointly regulated. The failure to identify any category as being statistically significantly associated with the genes identified suggests that DDP resistance may not be the result of co-opting canonical pathways but more a multifactorial phenomenon involving genes and proteins that have as yet uncharacterized behaviors.

Not all differentially expressed genes are expected to be causally linked to the resistant phenotype in ovarian cancer; however, certain new mechanistic insights are suggested by the data from the current study. Among the 27 genes of greatest interest, annexin A1, TIMP1, CLDN4, and STARD4 are cell-surface proteins involved in intercellular interactions. Drug resistance resulting from altered cell-cell contacts and gap junction communication between cells has previously been reported (Shain and Dalton, 2001; Croix et al., 1996). Loss of an adherens junction protein, β-catenin, presumably through proteolytic degradation, has also recently been associated with DDP resistance (Liang and Shen, 2004). As loss of cell-cell contacts (anoikis) has been noted to induce apoptosis (Frisch and Francis, 1994), increased contact through tight junctions as a result of up-regulation of component proteins such as CLDN4 may favor survival. Recently, the sharing of a ‘death signal’ instigated by the Ku70/Ku80/DNA-dependent protein kinase complex and transmitted from DDP-damaged cells to its neighbors through gap junctions been demonstrated (Jensen and Glazer, 2004). The known function of claudin 4 in regulating ion permeabilities (van Itallie and Fanning, 2003) may likewise directly modulate ‘death’ or survival signals.

There is a possible connection between resistance effects at the cell surface and the interior of the cell. In addition to known roles in mediating apoptosis, regulating cell adhesion molecules and signaling, annexin 1 (ANXA1) has been linked to resistance to doxorubicin, melphalan, and etoposide in MCF-7 cells via a mechanism that is independent of MRP1 and PgP1 but which is putatively linked to enhanced vesicle aggregation (Wang et al., 2004). The preponderance of other lipid metabolism and vesicle trafficking genes among the 27 genes identified in 4 of 6 cell pairs suggests their involvement in resistance mechanisms. STARD4 is a ubiquitous, cholesterol-regulated member of a family of homologous steroidogenic acute regulatory (STAR)-related lipid transfer proteins that shuttles lipids and sterols intracellularly (Soccio et al., 2002). Tissue factor has been shown to be regulated by endocytosis through at least two pathways, one of which is dependent on low-density lipoprotein receptor-related proteins such as ApoE. LDL-receptors have cysteine-rich modules and binding of platinum to sulfur moieties is well known. Finally, the data suggest alterations in cytoskeletal genes in DDP-resistant cells. Thymosin beta 10 has been implicated in actin reorganization and is associated with apoptosis (Lee et al., 2001). Annexin 1 has been linked to disruption of the actin cytoskeleton through sustained activation of the ERK signaling cascade and inhibition of cyclin D1 expression resulting in reduced cell proliferation (Alldridge and Bryant, (2003) ApoE signalling in neurons is associated with microtubule depolymerization (Beffert and Stolt, 2004). Furthermore, during the endocytotic recycling of tissue factor-Factor VIIa complexes, Factor VIIa is released from vesicles and is observed to bind to the actin cytoskeleton.

The expression profiling approach used in this study was successful in identifying differentially expressed genes and in providing an unusually high degree of confidence compared to studies based solely on comparison of unrelated cell lines or tumors. To attain this degree of confidence many more technical and biological replicates were required than are conventionally used. The high confidence that the genes identified are really differentially expressed as detected by microarray-based expression profiling suggests these genes as novel markers of DDP resistance in ovarian cancer. Although the differentially expressed genes described in this work do not map to known biochemical pathways or function ontology classifications, they do constitute a set of previously unidentified markers of DDP resistance in ovarian cancer that suggest here-to-fore undescribed mechanisms of resistance that may serve to predict DDP resistance in clinical samples.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,195
U.S. Pat. No. 4,683,202
U.S. Pat. No. 4,800,159
U.S. Pat. No. 4,883,750
U.S. Pat. No. 4,946,773
U.S. Pat. No. 5,279,721
U.S. Pat. No. 5,578,832
U.S. Pat. No. 5,837,832
U.S. Pat. No. 5,837,860
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,242
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
Alldridge and Bryant, Exp. Cell Res., 290: 93-107, 2003.
Andrews and Howell, Cancer Cells, 2: 35-43, 1990.
Bahr et al., J. Mass. Spectrom., 32: 1111, 1997.
Bahr et al., J. Mass. Spectrom., 32: 1111, 1997.
Baruah et al, Curr. Top Med. Chem., 4(15): 1537-1549, 2004.
Beffert et al., J. Lipid Res., 45: 403-409, 2004.
Belani, Semin. Oncol., 31(6 Suppl 14): 25-33, 2004.
Bellus, J. Macromol. Sci. Pure Appl. Chem., A31(1): 1355-1376, 1994.
Bentzley et al., Anal. Chem., 68(13): 2141-2146, 1996.
Blondal et al., Nucleic Acids Res., 31(24): e155, 2003.
Broude, Expert Rev. Mol. Diagn., 2(3): 209-216, 2002.
Bustin, J. Mol. Endocrinol., 29(1):23-39, 2002.
Cole et al., Science, 258: 1650-1654, 1992.
Cole, Cancer Res., 54: 357-361, 1994.
Croix et al., J. Natl. Cancer Inst., 88: 1285-1296, 1996.
Duncan et al., Rapid Commun. Mass Spectrom., 7: 1090, 1993.
Erlandsson, Cancer Genet. Cytogenet., 104(1): 1-18, 1998.
European Appln. 320 308
European Appln. 329 822
Farrell, Semin Oncol., 31(6 Suppl 14): 1-9, 2004.
Faulstich et al., Anal. Chem., 69(21): 4349-4353, 1997.
Faulstich et al., Anal. Chem., 69(21): 4349-4353, 1997.

Fodor et al., Biochemistry, 30(33): 8102-8108, 1991.
Frisch and Francis, J. Cell Biol., 124: 619-626, 1994.
Frohman, In: PCR Protocols: A Guide To Methods And Applications, Academic Press, N.Y., 1990.
GB Appln. 2 202 328
Gobom et al., Anal. Chem. 72: 3320, 2000.
Gobom et al., Anal. Chem. 72: 3320, 2000.
Godwin et al., Proc. Natl. Acad. Sci. USA, 89: 3070-3074, 1992.
Gros et al., Nature, 323: 728-731, 1986.

Hacia et al., Nature Genet., 14: 441-449, 1996.
Hall et al., Met. Ions Biol. Syst., 42: 297-322, 2004.
Holford et al., Eur. J. Cancer, 36: 1984-1990, 2000.

Holmstrom et al., Anal. Biochem. 209: 278-283, 1993.
Ihaka and Gentleman, J. Comput. Graph. Stats., 5: 299-314, 1996.
Innis et al., Proc. Natl. Acad. Sci. USA, 85(24): 9436-9440, 1988.
Jacobs et al., Clin. Cancer Res., 11(4): 1669-1674, 2005.
Jensen and Glazer, Proc. Natl. Acad. Sci. USA, 101: 6134-6139, 2004.
Jiang et al., J. Agric. Food Chem., 48: 3305, 2000.
Johnson and Hamdy, Oncol. Rep., 5(3): 553-557, 1998.
Kelley and Rozencweig, Eur. J. Clin. Oncol., 25: 1135-1140, 1989.
Kelley et al., Science, 241: 1813-1815, 1988.
Kolmel, J. Neurooncol., 38(2-3): 121-125, 1998.
Koropatnick and Pearson, Molec. Pharmacol., 44: 44-50, 1993.
Kwoh et al., Proc. Natl. Acad. Sci. USA, 86: 1173, 1989.
Lee et al., Oncogene, 20: 6700-6706, 2001.
Liang et al., Mol. Pharmacol., 65: 1217-1224, 2004.
Liang, Biotechniques, 33(2): 338-344, 346, 2002.
Liebermann, Oncogene, 17(10): 1189-94, 1998.
Magi-Galluzzi et al., Anal. Quant. Cytol. Histol., 20(5): 343-350, 1998.
Mangray and King, Front Biosci., 3: D1148-1160, 1998.
Marvin et al., Clin. Chim. Acta., 337(1-2): 11-21, 2003.
Miketova and Schram, Mol. Biotechnol., 8(3): 249-253, 1997.
Mirgorodskaya et al., Rapid Commun. Mass Spectrom., 14: 1226, 2000.
Mishima et al., Eur. J. Cancer, 38: 1405-1412, 2002.
Miyazaki et al., Biochem. Biophys. Res. Commun., 166: 1358-1364, 1990.
Morton et al., J. Pharmacol. Exp. Ther., 267: 697-702, 1993.
Mougin et al., Ann. Biol. Clin., (Paris) 56(1): 21-8, 1998.
Muddiman et al., Fres. J. Anal. Chem., 354: 103, 1996.
Mumby and Walter, Cell Regul., 2(8): 589-98, 1991.
Natile et al, Met. Ions Biol. Syst., 42: 209-250, 2004.
Natoli et al., Biochem. Pharmacol., 56(8): 915-20, 1998.
Nelson et al., Anal. Chem., 66: 1408, 1994.
Newton et al., Nucl. Acids Res. 21: 1155-1162, 1993.
Ochi et al., Am. J. Gastroenterol., 93(8): 1366-1368, 1998.
Ohara et al., Proc. Natl. Acad. Sci. USA, 86: 5673-5677, 1989.
PCT Appln. PCT/US89/01025
PCT Appln. WO 88/10315
PCT Appln. WO 89/06700
PCT/US87/00880
Pease et al., Proc. Natl. Acad. Sci. USA, 91: 5022-5026, 1994.
Perez et al., Pharmacol. Ther., 48: 19-27, 1990.
Puchalski et al., Proc. Natl. Acad. Sci. USA, 87: 2443-2447, 1990.
Pusch et al., Pharmacogenomics, 4(4): 463-476, 2003.
Rasmussen et al., Anal. Biochem, 198: 138-142, 1991.
Riordan et al., Nature, 316: 817-819, 1985.
Roninson et al., Nature, 309: 626-628, 1984.
Running et al., BioTechniques 8: 276-277, 1990.
Sambrook et al., In: Molecular cloning, Cold Spring Harbor Laboratory Press, Cold Spring Harbor, N.Y., 2001.
Sauer and and Gut, J. Chromatogr. B Analyt. Technol. Biomed. Life Sci., 782(1-2): 73-87, 2002.
Shain and Dalton, Mol. Cancer Ther., 1: 69-78, 2001.
Shoemaker et al., Nature Genetics, 14: 450-456, 1996.
Soccio et al., Proc. Natl. Acad. Sci. USA, 99: 6943-6948, 2002.
Solyanik et al., Cell. Prolif., 28(5): 263-278, 1995.
Stein and Liang, Cell Mol. Life Sci., 59(8): 1235-1240, 2002.
Sternberg et al, Oncology, 68(1): 2-9, 2005.
Stokke et al., Cell. Prolif., 30(5): 197-218, 1997.
Storey and Tibshirani, In: In: The analysis of gene expression data: methods and software, Springer, NY, 2003.
Timmer-Bosscha et al., Br. J. Cancer, 66: 227-238, 1992.
Tost and Gut, Mass Spectrom. Rev., 21(6): 388-418, 2002.
Tusher et al., Proc. Natl. Acad. Sci. USA, 98: 5116-21, 2001.
Van Itallie et al., Am. J. Physiol. Renal. Physiol., 285: F1078-1084, 2003.
Vogelstein and Kinzler, Cell, 70(4): 523-6, 1992.
Walker et al., Proc. Natl. Acad. Sci. USA, 89: 392-396, 1992.
Wang et al., Biochem. Biophys. Res. Commun., 314: 565-570, 2004.
Wang et al., J. Agric. Food. Chem., 47: 1549, 1999.
Wang et al., J. Agric. Food. Chem., 47: 2009, 1999.
Wang et al., J. Agric. Food. Chem., 48: 2807, 2000.
Wang et al., J. Agric. Food. Chem., 48: 3330, 2000.
Wittmann et al., Biotechnol. Bioeng., 72: 642, 2001.
Wittmann et al., Biotechnol. Bioeng., 72: 642, 2001.
Wu et al., Biochem. Biophys. Res. Commun., 233(1): 221-6, 1997.
Xu et al., Mass Spectrom Rev., 22(6): 429-440, 2003.
Yang et al., FEBS Lett., 439: 368-372, 1998.
Yang et al., J. Agric. Food. Chem., 48: 3990, 2000.
Yang et al., Nucleic Acids Res., 30: 15-200, 2002.

What is claimed is:

1. A method of detecting resistance to a platinum-drug based therapy comprising assessing expression in a cell of at least one gene from group 1 or group 2; wherein group 1 comprises TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, PRO2000, GPR126, OSTF1, IMAGE:924929, IMAGE:79216, and IMAGE:1473168; and wherein group 2 comprises DPH2L1, ENDO180, IFITM1, RIMS1, MHC class II suppressor, IMAGE:868555, and IMAGE:1417815; wherein increased expression of a gene from group 1 or decreased expression of a gene from group 2 indicates that the cell is resistant to the platinum-drug based therapy.

2. The method of claim 1, wherein group 1 comprises TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, IMAGE:924929, IMAGE:79216, and IMAGE:1473168.

3. The method of claim 2, wherein group 2 comprises DPH2L1, ENDO180, IFITM1, IMAGE:868555, and IMAGE:1417815.

4. The method of claim 3, wherein the cell is a cancerous or pre-cancerous cell.

5. The method of claim 4, wherein the cell is a cancerous cell.

6. The method of claim 4, wherein the cell is a human cell.

7. The method of claim 6, wherein the cell is comprised in a subject.

8. The method of claim 7, wherein the subject is a human.

9. The method of claim 6, wherein the method further comprises extraction of RNA from said cell.

10. The method of claim 9, wherein expression of said gene is determined using northern blot, PCR, real-time PCR, RT-PCR, Q-RT-PCR, or differential display.

11. The method of claim 9, wherein expression of said gene is determined by assessing the amount of mRNA transcribed from the gene in the cell.

12. The method of claim 9, wherein expression of said gene is determined using a DNA chip or a microarray.

13. The method of claim 12, wherein the method further comprises determining the expression of at least a second gene whose expression may influence a cancer phenotype, wherein said second gene is not from group 1 or group 2.

14. The method of claim 6, wherein the method comprises determining the expression of at least two genes from group 1 or group 2.

15. The method of claim 14, wherein the method comprises determining the expression of at least three genes from group 1 or group 2.

16. The method of claim 15, wherein the method comprises determining the expression of at least four genes from group 1 or group 2.

17. The method of claim 16, wherein the method comprises determining the expression of at least five genes from group 1 or group 2.

18. The method of claim 17, wherein the method comprises determining the expression of all genes from group 1 and group 2.

19. The method of claim 6, wherein the method further comprises assessing the expression of metallothionein IIA in the cell.

20. The method of claim 8, wherein the method comprises individualization of a cancer therapy for the subject.

21. The method of claim 20, wherein the individualization of a cancer therapy comprises administering a chemotherapeutic, an anti-cancer drug, a surgical therapy, or a radiation therapy to the subject.

22. The method of claim 21, wherein the anti-cancer drug is Avastin or Herceptin.

23. The method of claim 20, wherein the individualization of a cancer therapy comprises administering a platinum-drug based chemotherapeutic to the subject.

24. The method of claim 23, wherein the individualization of a cancer therapy further comprises the administration of a second chemotherapeutic.

25. The method of claim 20, wherein the individualization of a cancer therapy comprises avoiding the administration of a platinum-drug based chemotherapeutic to the subject.

26. The method of claim 5, wherein the cancer originated in the brain, oral cavity, upper respiratory tract, lung, breast, upper gastrointestinal tract, lower gastrointestinal tract, pancreas, liver, kidney, bladder, prostate, bone, skin, bone marrow, a lymphatic organ, testis, ovary, Fallopian tube, or peritoneum.

27. The method of claim 5, wherein the cancer is ovarian cancer.

28. The method of claim 5, wherein the cell is comprised in a solid tumor.

29. The method of claim 5, wherein the cell is metastasized.

30. The method of claim 8, wherein the method comprises administration of the platinum-drug based therapy to the subject, and subsequently monitoring for resistance to the platinum-drug based therapy.

31. The method of claim 8, wherein the method comprises monitoring for resistance to the platinum-drug based therapy prior to the administration of a platinum-drug based therapy to the subject.

32. The method of claim 1, wherein the platinum-drug based therapy is carboplatin, oxaliplatin, or satraplatin.

33. The method of claim 1, wherein the platinum-drug based therapy is cisplatin.

34. A diagnostic kit for detecting resistance to a platinum-drug based therapy, said kit comprising probes for a group of one or more of the genes comprising TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, DPH2L1, ENDO180, IFITM1, IMAGE:924929, IMAGE:79216, IMAGE:1473168, IMAGE:868555, and IMAGE:1417815.

35. The kit of claim 34, wherein said probes are PCR primers for said group of genes.

36. The kit of claim 34, further comprises reagents for PCR reactions.

37. The kit of claim 34, wherein the platinum-drug based therapy is cisplatin.

38. A diagnostic array for detecting cisplatin resistance, said array comprises a solid support and a plurality of diagnostic agents coupled to said solid support, wherein said diagnostic agents are used to assay the expression levels of one or more of TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, DPH2L1, ENDO180, metallothionein IIA, IFITM1, IMAGE:924929, IMAGE:79216, IMAGE:1473168, IMAGE:868555, and IMAGE:1417815.

39. The diagnostic array of claim 12, wherein said diagnostic agents are DNA or RNA molecules that specifically hybridize to the transcripts of said genes.

40. A method for identifying a modulator of the expression of resistance to a platinum-drug based therapy comprising:
(a) providing a candidate modulator;
(b) contacting the candidate modulator with a cell;
(c) evaluating the expression of one or more of TXNIP, ANXA1, APOE, CLDN4, DKFZP564D0462, DKFZp761C121, F3, GADD45B, JUN, KIAA0470, MGC5254, OSTF1, PELI1, STARD4, TIMP1, TMSB10, DPH2L1, ENDO180, IFITM1, IMAGE:924929, IMAGE:79216, IMAGE:1473168, IMAGE:868555, or IMAGE:1417815 in the cell;
(d) comparing the expression measured in step (c) with the expression of the cell in the absence of said candidate modulator,
wherein a difference between the expression indicates that said candidate modulator can affect resistance to a platinum-drug based therapy.

41. The method of claim 40, wherein the platinum-drug based therapy is cisplatin.

42. The method of claim 40, wherein the cell is a cancerous cell.

43. The method of claim 42, wherein the cancerous cell is a human ovarian cancer cell.

44. The method of claim 40, wherein the method further comprises evaluating the expression of metallothionein IIA in the cell.
